(19) United States
(12) Patent Application Publication
(10) Pub. No.: US 2025/0082885 A1
(43) Pub. Date: Mar. 13, 2025

(54) PATIENT INTERFACE

(71) Applicant: ResMed Pty Ltd, Bella Vista, New South Wales (AU)

(72) Inventors: Lachlan Richard GOLDSPINK, Sydney (AU); Michiel Francis KOOIJ, Sydney (AU); Hugh Francis Stewart THOMAS, Sydney (AU)

(21) Appl. No.: 18/580,526

(22) PCT Filed: Jul. 20, 2022
(86) PCT No.: PCT/AU2022/050773

§ 371 Date: Jan. 18, 2024

(30) Foreign Application Priority Data
Jul. 20, 2021 (AU) 2021902228
Nov. 9, 2021 (AU) 2021903569

(51) Int. Cl. A61M 16/06; A61M 16/08

(52) U.S. Cl. CPC A61M 16/0683; A61M 16/0616; A61M 16/0883

(57) ABSTRACT

A patient interface comprises a frame which defines, at least in part, a plenum chamber, and a positioning and stabilising structure comprising at least one headgear tube. The frame, at least one headgear tube and manifold portion are integrally formed. In examples, no part of the frame or headgear tube has a Shore A Durometer hardness greater than 80. A method of manufacturing a component for a patient interface from a textile is also disclosed.

### 1.1 FIELD OF THE TECHNOLOGY

The present technology relates to one or more of the screening, diagnosis, monitoring, treatment, prevention and amelioration of respiratory-related disorders. The present technology also relates to medical devices or apparatus, and their use.

### 1.2 DESCRIPTION OF THE RELATED ART

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate”

### 1.2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.2.4 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Patient interfaces often consist of a plurality of separately manufactured but connected components. For example, a patient interface may comprise a cushion connected to a frame, with headgear also connected to the frame. The cushion may be made from a silicone membrane while the frame may comprise a hard polycarbonate. In some examples the headgear may comprise elastic straps while in other examples the headgear may comprise silicone tubes, as described further below.

Typically, where any two components are connected together, at least one of the components must comprise a relatively rigid connecting portion to allow the components to be connected securely. In one example the cushion may comprise a connector component made from a hard plastic that connects to the frame in a snap fit. Frames may also be provided with hard connectors for engaging headgear tubes. The presence of components comprising hard materials may detract from the comfort of the patient interface, may increase the likelihood of leaks, and/or may limit the extent to which the patient interface can be stored in a folded configuration, for example during shipping.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being one or more of obtrusive, aesthetically undesirable, costly, poorly fitting, difficult to use, and/or uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.2.4.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.2.4.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 1.2.2.4.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

1.2.2.4.4 Pressurised Air Conduit used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head. In one example the headgear tubing extends on both sides of the patient's face to the top of the patient's head. In another example one of the tubes is replaced by a strap.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

One factor which may make the headgear of some mask designs less comfortable and/or may detract from the stability of the seal with the patient's face is the presence of rigid connectors between the headgear tubing and the plenum chamber. Such connectors tend to be relatively bulky. The size and rigidity of the connectors increases the likelihood that external forces on the connector (for example caused by the connector coming into contact with a pillow) tend to be transferred to the seal forming structure, potentially disrupting the seal. Such connectors may also transfer force into the patient's face, which may be uncomfortable.

In other example, it may be desirable to provide a layer of material, for example a textile, between the tubing and the patient, at least at points where the tubing would otherwise touch the skin.

### 1.2.2.5 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

1.2.2.6 Air circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.2.7 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.2.8 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

1.2.2.9 Vent technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:2007, 10 cmH2O pressure at 1 m)

| | | A-weighted | A-weighted | |
|---|---|---|---|---|
| | | sound power | sound pressure | |
| | | level dB(A) | dB(A) | |
| Mask name | Mask type | (uncertainty) | (uncertainty) | Year (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage ™ (*) | | | | |
| ResMed | nasal | 36 (3) | 28 (3) | 2000 |
| UltraMirage ™ | | | | |
| ResMed | nasal | 32 (3) | 24 (3) | 2002 |
| Mirage | | | | |
| Activa ™ | | | | |
| ResMed | nasal | 30 (3) | 22 (3) | 2008 |
| Mirage | | | | |
| Micro ™ | | | | |
| ResMed | nasal | 29 (3) | 22 (3) | 2008 |
| Mirage ™ | | | | |
| SoftGel | | | | |
| ResMed | nasal | 26 (3) | 18 (3) | 2010 |
| Mirage ™ FX | | | | |
| ResMed | nasal pillows | 37 | 29 | 2004 |
| Mirage Swift ™ | | | | |
| (*) | | | | |
| ResMed | nasal pillows | 28 (3) | 20 (3) | 2005 |
| Mirage Swift ™ | | | | |
| II | | | | |
| ResMed | nasal pillows | 25 (3) | 17 (3) | 2008 |
| Mirage Swift ™ | | | | |
| LT | | | | |
| ResMed AirFit | nasal pillows | 21 (3) | 13 (3) | 2014 |
| P10 | | | | |

(* one specimen only, measured using test method specified in ISO 3744 in CPAP mode at 10 cmH2O)

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, case of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a patient interface comprising a frame which defines, at least in part, a plenum chamber, and a positioning and stabilising structure comprising a pair of headgear tubes and a manifold portion connected to both tubes, wherein the frame, headgear tubes and manifold portion are integrally formed.

In examples: a) the patient interface comprises a seal support portion which is formed integrally with the frame; b) the patient interface is provided with a seal portion, wherein the seal portion is connected to the seal support portion; c) the seal portion is releasably connected to the seal support portion; d) the patient interface comprises a seal forming structure which is formed integrally with the frame; e) the seal forming structure comprises a textile layer; f) the textile layer is provided with a silicone backing layer; g) the patient interface comprises a flexible cover which covers at least portions of each tube; h) the flexible cover comprises a textile; i) the flexible cover comprises at least one opening configured to allow inspection of the tube through the opening; j) the flexible cover comprises a strap connecting structure for connecting a back strap to the flexible cover; k) the flexible cover comprises a back strap; 1) a back strap is integrally formed with the flexible cover; m) the patient interface comprises a vent module; n) the vent module is configured to engage the frame in a stretch fit; o) the patient interface comprises a heat and moisture exchanger connected to the vent module; and/or p) the patient interface comprises a heat and moisture exchanger.

Another form of the present technology comprises a patient interface comprising a frame which defines, at least in part, a plenum chamber, and a positioning and stabilising structure comprising at least one headgear tube, wherein the frame, at least one headgear tube and manifold portion are integrally formed.

In examples: a) the patient interface comprises a seal support portion which is formed integrally with the frame; b) the patient interface is provided with a seal portion, wherein the seal portion is connected to the seal support portion; c) the seal portion is releasably connected to the seal support portion; d) the patient interface comprises a seal forming structure which is formed integrally with the frame; e) the seal forming structure comprises a textile layer; f) the textile layer is provided with a silicone backing layer; g) the patient interface comprises a flexible cover which covers at least portions of each tube; h) the flexible cover comprises a textile; i) the flexible cover comprises at least one opening configured to allow inspection of the tube through the opening; j) the flexible cover comprises a strap connecting structure for connecting a back strap to the flexible cover; k) the flexible cover comprises a back strap; 1) a back strap is integrally formed with the flexible cover; m) the patient interface comprises a vent module; n) the vent module is configured to engage the frame in a stretch fit; o) the patient interface comprises a heat and moisture exchanger connected to the vent module; and/or p) the patient interface comprises a heat and moisture exchanger.

Another form of the present technology comprises a patient interface comprising a frame which defines, at least in part, a plenum chamber, and at least one headgear tube, wherein no part of the frame or headgear tube has a Shore A Durometer hardness greater than 80.

Another form of the present technology comprises a patient interface comprising a frame which defines, at least in part, a plenum chamber, and at least one headgear tube, wherein the frame and headgear tube are made from the same material, and wherein the material has a Shore A Durometer hardness of no more than 80.

Another form of the present technology comprises a component for a patient interface comprising a vent module connected to a heat and moisture exchanger, the vent module comprising at least one vent, the vent module further comprising a formation which is configured to removably engage a frame of a patient interface.

In examples: a) the formation comprises a channel configured to receive a rim of an opening in the frame of a patient interface in a stretch fit; b) the heat and moisture exchanger comprises open cell reticulated polyester foam; c) the component is configured for use with a patient interface having a plenum chamber having a predetermined size and shape; and/or d) the heat and moisture exchanger is configured to fill more than 90% of a volume of the plenum chamber.

Another form of the present technology comprises a patient interface comprising a frame which defines, at least in part, a plenum chamber, and at least one headgear tube, wherein no connector is provided between the frame and the at least one headgear tube.

Another form of the present technology comprises a patient interface comprising a frame which defines, at least in part, a plenum chamber, and a positioning and stabilising structure comprising at least one headgear tube, wherein the frame and at least one headgear tube are integrally formed,
the patient interface further comprising a flexible cover configured to cover the frame and at least a portion of the tube,
wherein the flexible cover comprises a textile and a seal forming portion of the flexible cover is constructed and arranged to form a seal around and allow a flow of air to at least an entrance to the patient's nares,
the patient interface further comprising a vent.

In examples: a) the frame defines an opening in a superior face thereof, the opening extending to a posterior side of the frame, wherein the frame is configured to hold the seal forming portion taut across the opening; b) a rim of the opening is resiliently flexible; c) the rim of the opening comprises a plurality of slits; d) the slits form an angle of between 70 degrees and 90 degrees to the opening; e) portions of the rim between the slits can deflect independently of each other; f) each portion of the rim between the slits is configured to function as a cantilever spring element; g) the frame comprises posteriorly extending support portions which are configured to bring the seal forming portion into engagement with regions of the patient's face proximate the alar creases; h) the posteriorly extending support portions are stiffer than the rim; i) the posteriorly extending portions comprise a stiffer and/or thicker material than the material of the rim; j) the frame comprises at least one internal rib to stiffen the posteriorly extending support portions; k) the frame and/or at least one headgear tube comprise at least one indexing structure configured to engage one or more preselected portions of the flexible cover to thereby set the orientation of the seal forming portion relative to the frame; 1) the indexing structure comprise at least one protuberance on the frame and/or headgear tube; and/or m) the preselected portion of the flexible cover comprises a slit or aperture in the cover.

Another form of the present technology comprises a substructure for a patient interface, the patient interface comprising a flexible cover comprising a textile, the flexible cover further comprising a seal forming portion which is constructed and arranged to form a seal around and allow a flow of air to at least an entrance to the patient's nares,
the substructure comprising:
a frame which defines, at least in part, a plenum chamber; and
a positioning and stabilising structure comprising at least one headgear tube,
wherein the at least one headgear tube is formed integrally with the frame,
wherein the frame defines an opening in a superior face thereof, the opening extending to a posterior side of the frame, and
wherein the frame is configured to hold the seal forming portion taut across the opening when in use.

Another form of the present technology comprises a patient interface comprising a substantially air-tight textile structure which defines, at least in part, a plenum chamber, the textile structure further defining a seal forming structure constructed and arranged to form a seal around at least an entrance to the patient's nares, the textile structure further comprising at least one headgear tube, the patient interface further comprising an undercushion configured to be removably positioned within the plenum chamber, the undercushion configured to support lateral and anterior edges of the seal forming structure, the patient interface further comprising a vent.

In examples: a) the undercushion comprises a chassis portion having an opening in a superior face thereof, the opening extending to a posterior side of the chassis portion, wherein the chassis portion is configured to hold a seal forming portion of the textile structure taut across the opening; b) the chassis portion comprises posteriorly extending support portions which are configured to bring the seal forming portion into engagement with regions of the patient's face proximate the alar creases; c) the chassis portion comprises a plurality of channels, wherein the channels are configured to allow air from the headgear tubes to enter the plenum chamber; d) the channels are configured to allow flow from the seal forming portion through the plenum chamber to a vent module; e) the undercushion comprises a heat and moisture exchange portion provided to the opening in the chassis portion, wherein a superior surface of the heat and moisture exchange material is below a rim of the opening; and/or f) the channels are configured to allow flow from the headgear tubes to flow to the vent without passing through the heat and moisture exchange material.

Another form of the present technology comprises an undercushion for a patient interface, the patient interface comprising a textile structure which defines, at least in part, a plenum chamber, the textile structure further defining a seal forming structure and at least one headgear tube,
wherein the undercushion is configured to be positioned within the plenum chamber to support lateral and anterior edges of the seal forming structure, wherein the undercushion comprises a body which is separate from, and made from a different material to, the textile structure.

In examples, the undercushion is made from foam.

Another form of the present technology comprises a frame and at least one headgear tube integrally formed with the frame, the patient interface further comprising a cushion module configured for removable connection to the frame, the cushion module defining a plenum chamber, the cushion module comprising an undercushion and a seal forming structure comprising a membrane covering the undercushion, the undercushion configured to support lateral and anterior edges of the seal forming structure, wherein a portion of the membrane between the lateral edges is not supported by the undercushion;
the seal forming structure constructed and arranged to form a seal around, and allow a flow of air to, at least an entrance to the patient's nares,
the cushion module further comprising at least one inlet port to receive a flow of air from the at least one headgear tube,
the patient interface further comprising a vent.

In examples: a) the undercushion comprises posteriorly extending support portions which are configured to bring the seal forming structure into engagement with regions of the patient's face proximate the alar creases; b) the cushion module comprises a heat and moisture exchanger; c) a posterior side of the frame defines a concave shape and an anterior side of the cushion module defines a complementary convex shape; d) the frame is substantially U shaped; e) the cushion module comprises a pair of inlet ports; f) the frame comprises a pair of boss portions configured to engage the inlet ports and secure the cushion module to the frame; g) each boss portion comprises an aperture therethrough to allow flow from a respective headgear tube to a respective inlet port; and/or h) the cushion module comprises a vent and the frame comprises at least one vent aperture to allow flow from the vent to atmosphere.

Another form of the present technology comprises a cushion module for a patient interface, the cushion module configured for removable connection to a frame of the patient interface, the cushion module defining a plenum chamber, the cushion module comprising an undercushion and a seal forming structure comprising a membrane covering the undercushion, the undercushion configured to support lateral and anterior edges of the seal forming structure and to leave a portion of the membrane between the edges unsupported;
the seal forming structure constructed and arranged to seal around, and allow a flow of air to, at least an entrance to the patient's nares,
the cushion module further comprising at least one inlet port to receive a flow of air from the at least one headgear tube,
wherein the inlet ports of the cushion module are configured to receive respective boss portions of the frame to thereby engage the cushion module with the frame in use.

According to one form of the technology there is provided a method of manufacturing a component for a patient interface comprising the steps of:
i) forming a textile blank;
ii) inserting the textile blank into a female mould, wherein the female mould comprises a plurality of apertures configured to allow application of a vacuum to the outer surface of the textile blank when the blank is in the mould;
iii) applying a vacuum to the apertures in the mould to hold the textile blank in a required shape; and
iv) spraying a settable material on an inner surface of the textile blank.

In examples: a) the settable material is air impervious when set; b) the settable material is silicone; c) the step of spraying the settable material comprises the steps of i) inserting a flexible tube provided with a spray nozzle into the textile blank; and ii) withdrawing the flexible tube and spray nozzle from the textile blank while spraying the settable material; d) the method comprises spraying a plurality of layers of settable material to the inner surface of the blank; e) the required shape is different to the shape of the blank; f) the component comprises a tube; g) the tube is a corrugated tube; h) the textile blank is not corrugated prior to insertion into the female mould, and the mould is configured to hold the textile blank in a corrugated configuration during the step of spraying the settable material; and/or i) the component comprises a plenum chamber, a frame and a seal forming structure.

According to one form of the technology there is provided a patient interface comprising a plenum chamber pressurisable to a therapeutic pressure of at least 6 cmH2O above ambient air pressure, said plenum chamber including a plenum chamber inlet port sized and structured to receive a flow of air at the therapeutic pressure for breathing by a patient,
a seal-forming structure constructed and arranged to form a seal with a region of the patient's face surrounding an entrance to the patient's airways, said seal-forming structure having a plurality of holes therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a positioning and stabilising structure to provide a force to hold the seal-forming structure in a therapeutically effective position on the patient's head, the positioning and stabilising structure comprising a tie, the tie being constructed and arranged so that at least a portion overlies a region of the patient's head superior to an otobasion superior of the patient's head in use; and
a vent structure to allow a continuous flow of gases exhaled by the patient from an interior of the plenum chamber to ambient, said vent structure being sized and shaped to maintain the therapeutic pressure in the plenum chamber in use;
wherein
the patient interface is configured to allow the patient to breath from ambient through their mouth in the absence of a flow of pressurised air through the plenum chamber inlet port, or the patient interface is configured to leave the patient's mouth uncovered; and
wherein the plurality of holes comprises at least four holes.

In examples: a) the plurality of holes comprises at least 16 holes; b) the plurality of holes comprises at least 46 holes; c) the plurality of holes comprises 122 holes; d) the plurality of holes are formed in two groups, wherein each group is configured to communicate with a respective one of the patient's nares in use; and/or e) a strip is provided between the two groups of holes, wherein no holes are provided in the strip.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

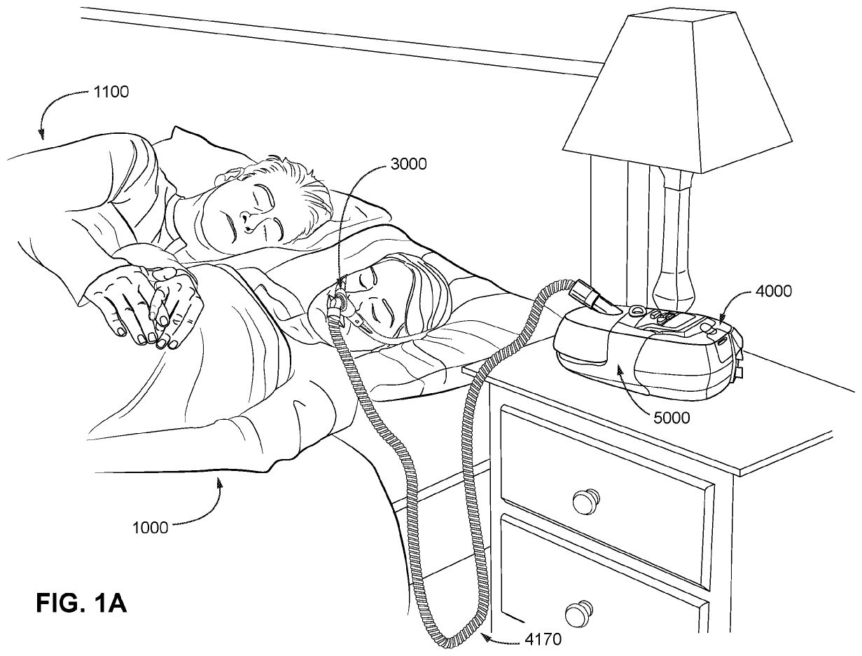

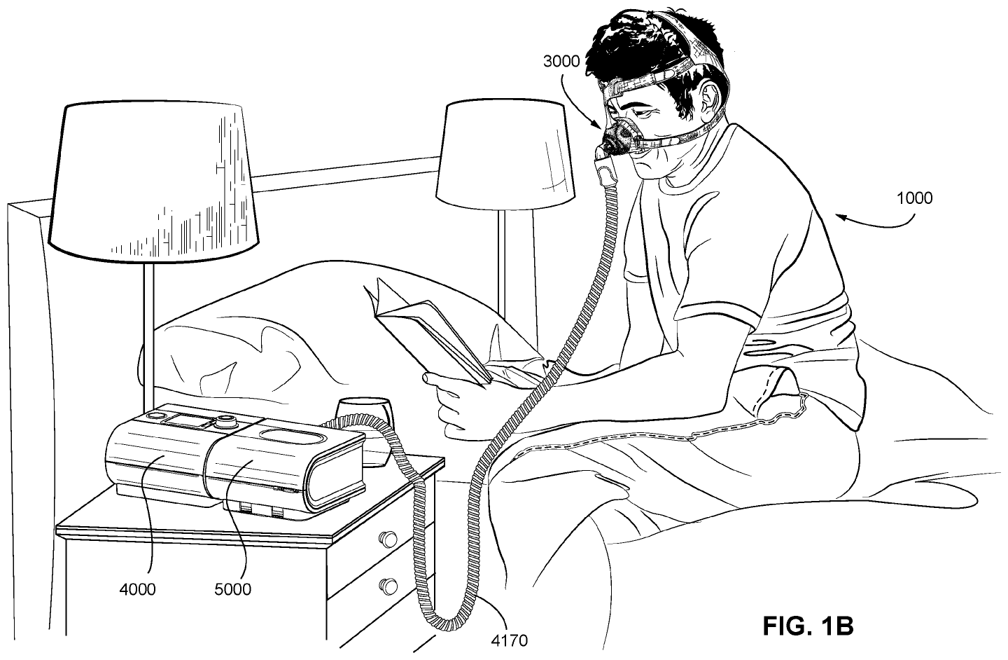

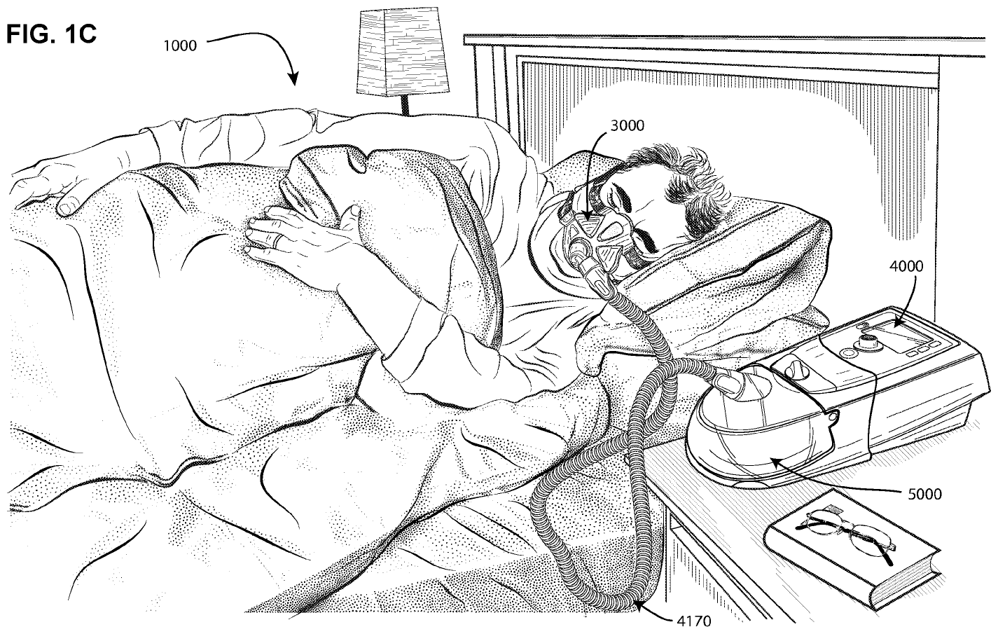

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

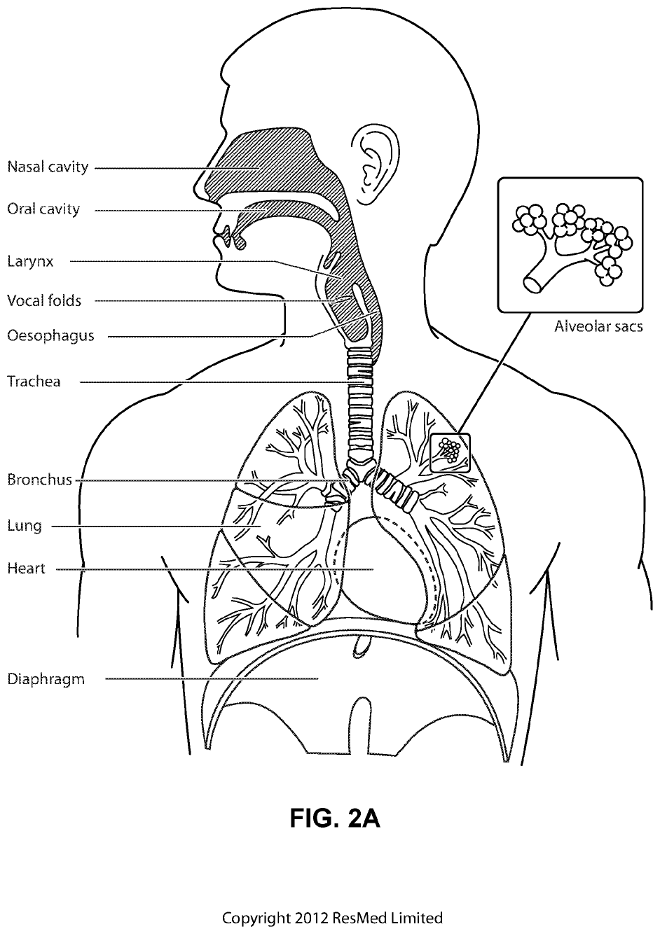

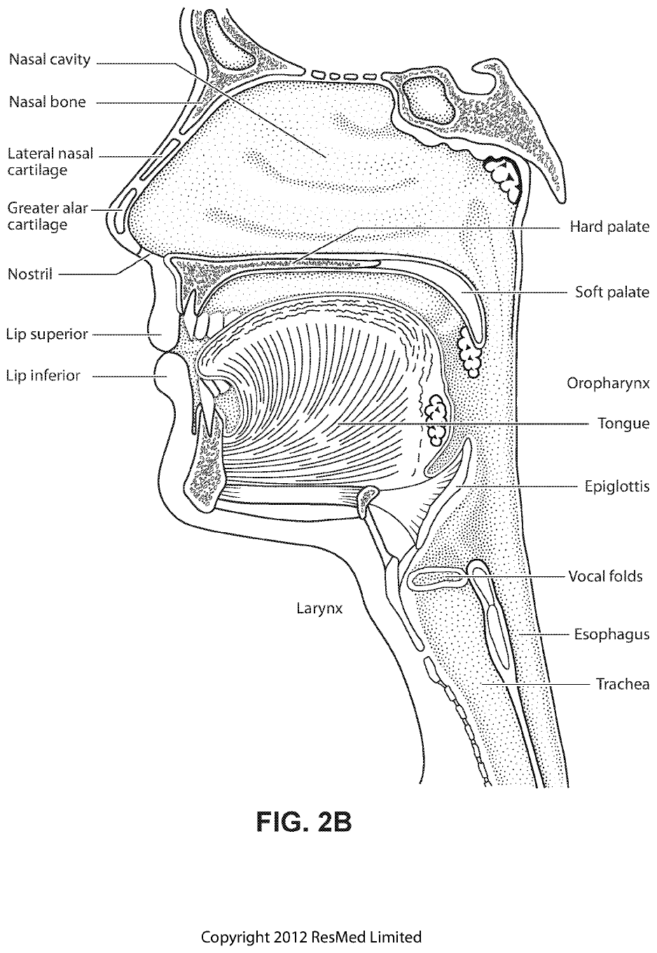

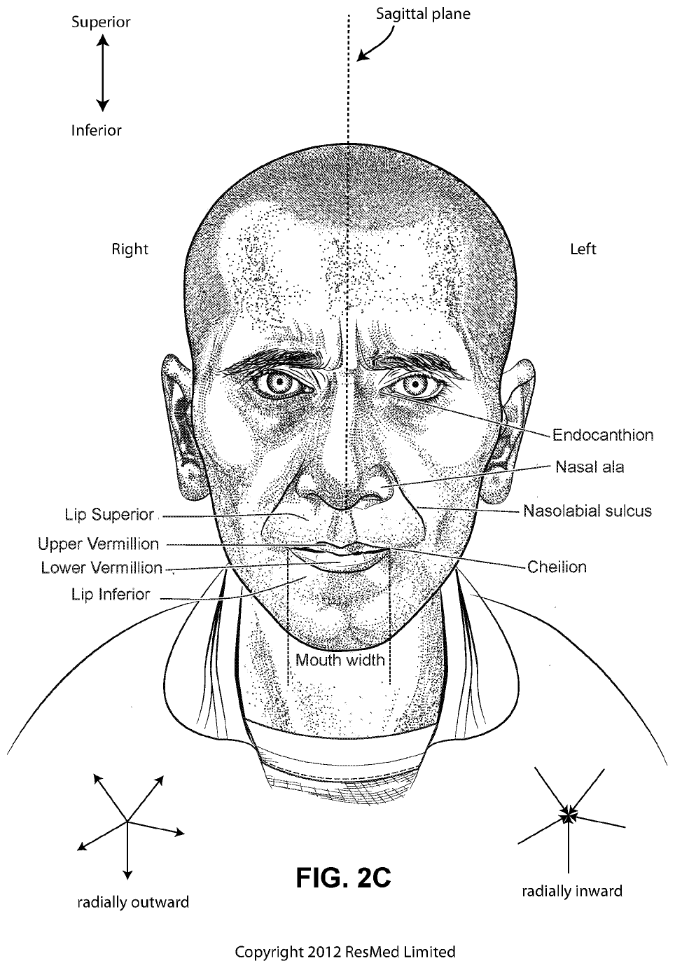

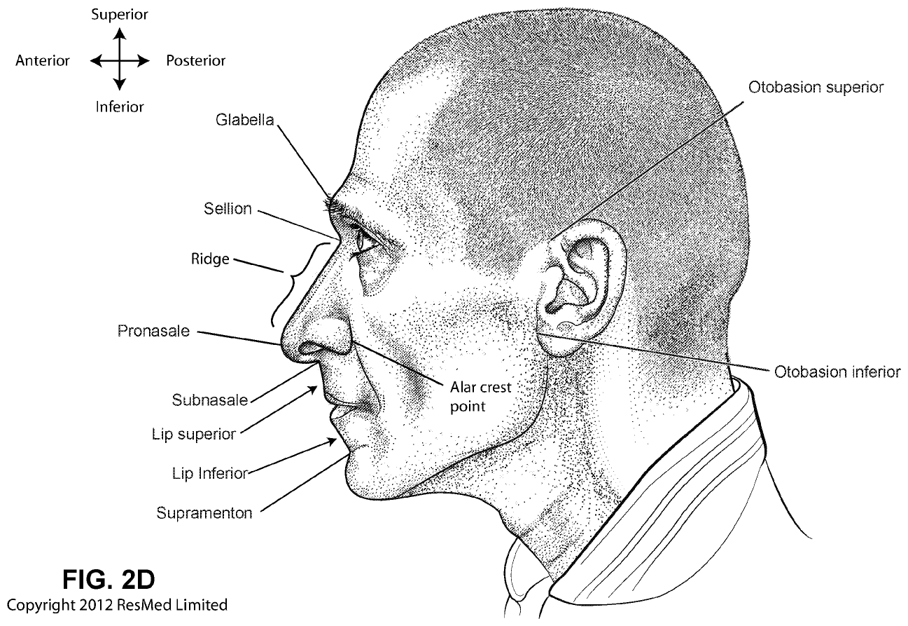

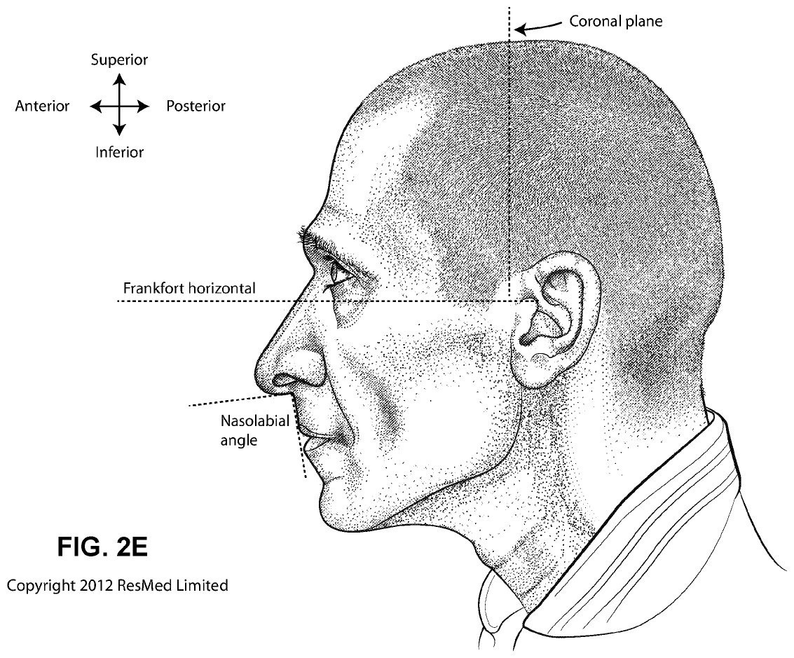

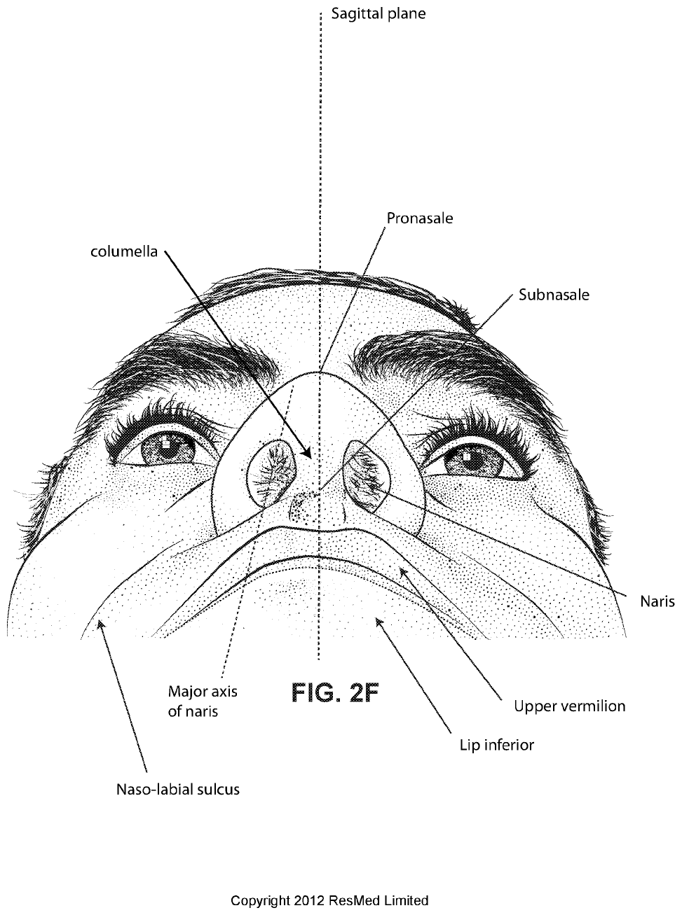

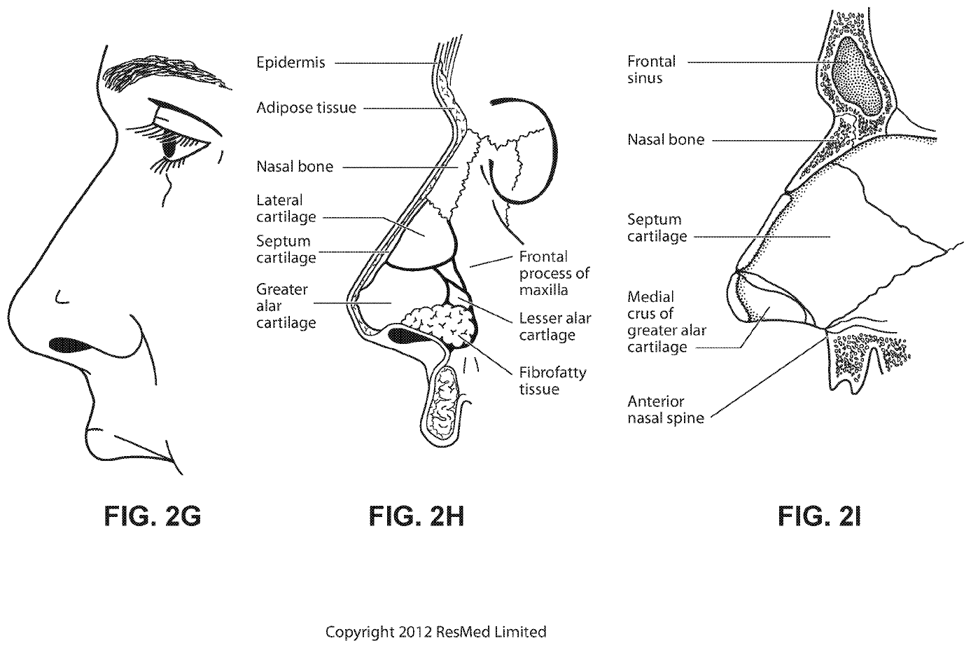

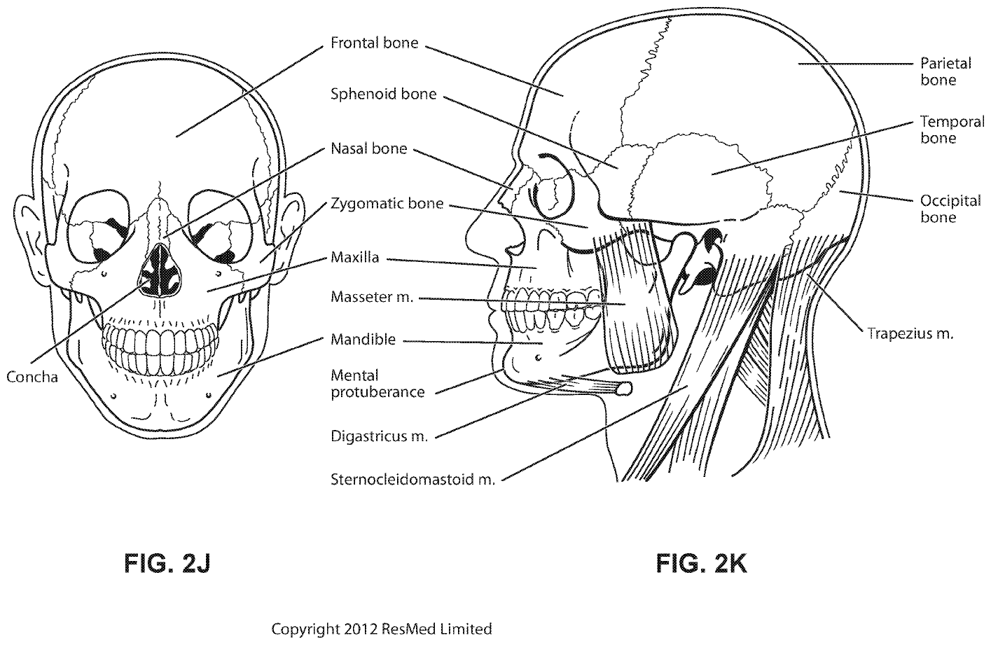

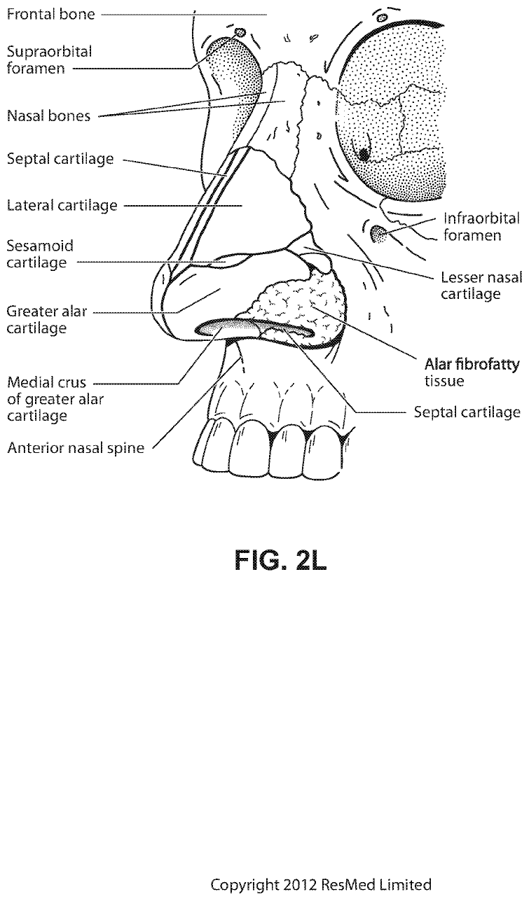

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideo trapezius.

FIG. 2L shows an anterolateral view of a nose.

### 3.3 Patient Interface

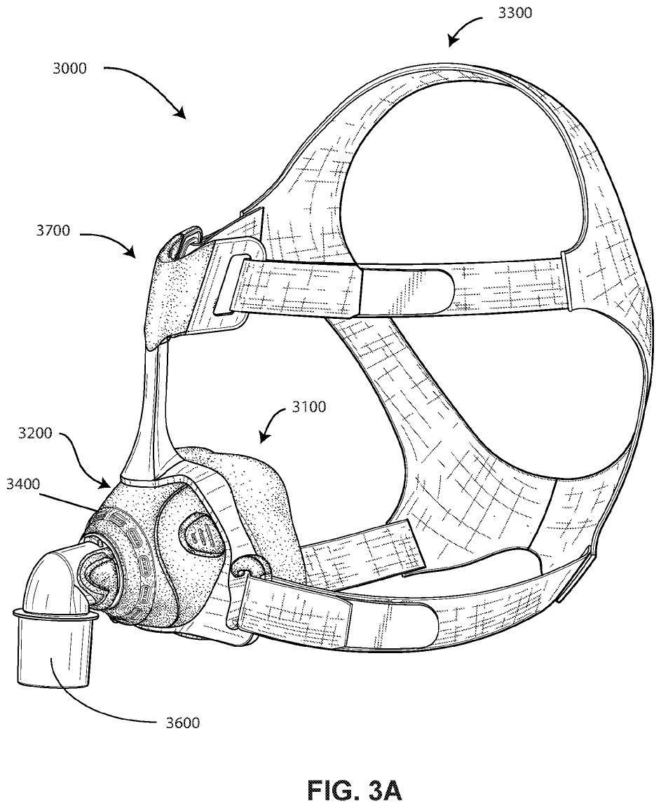

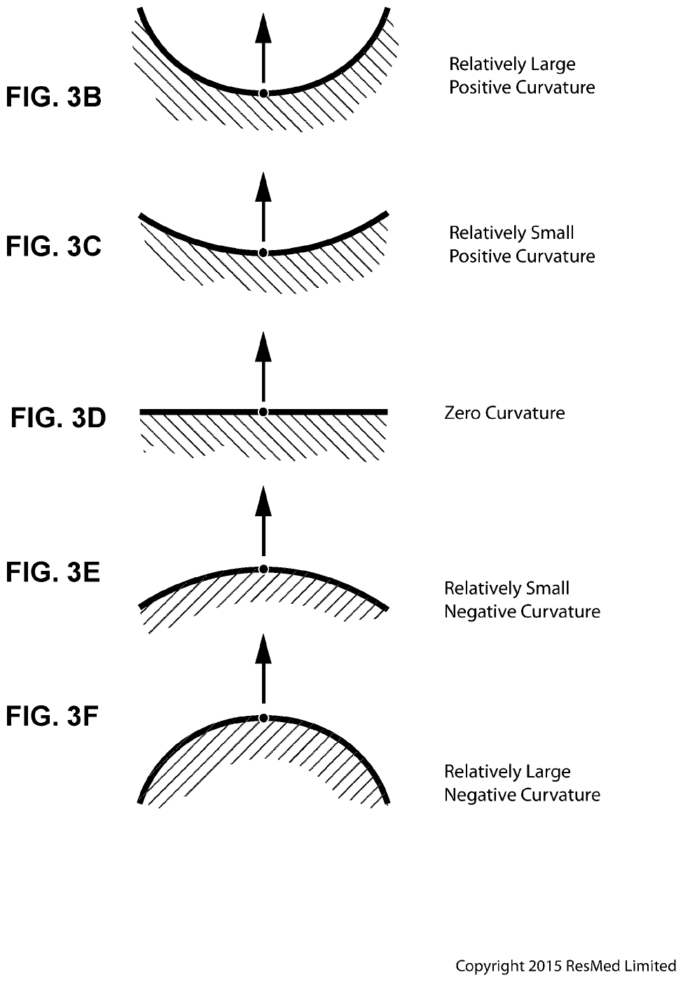

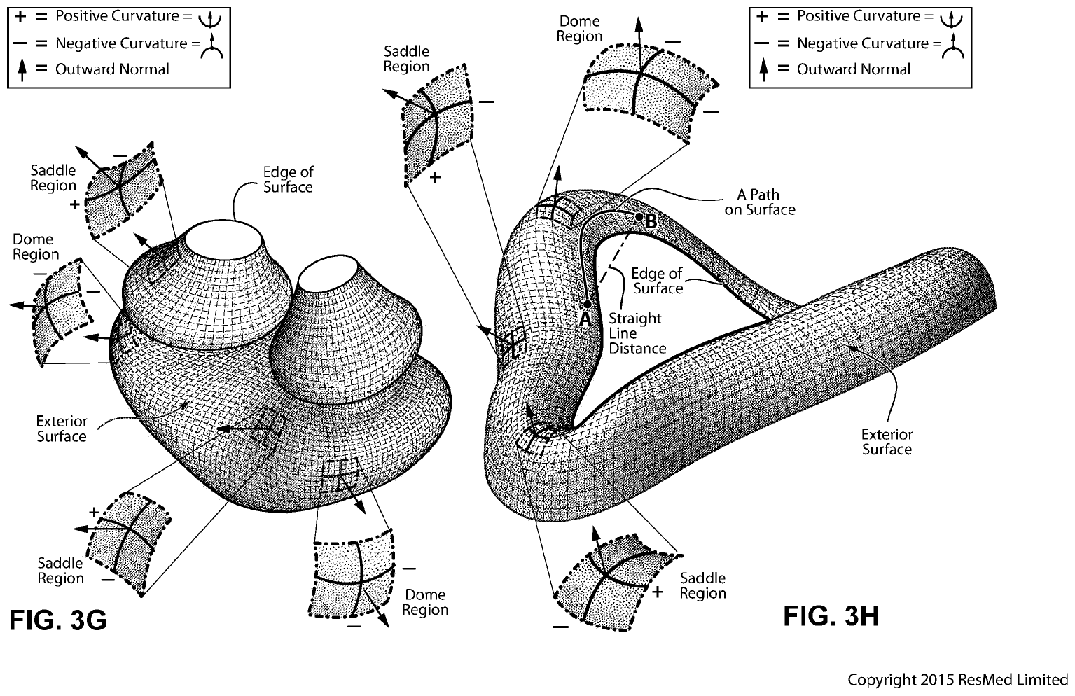

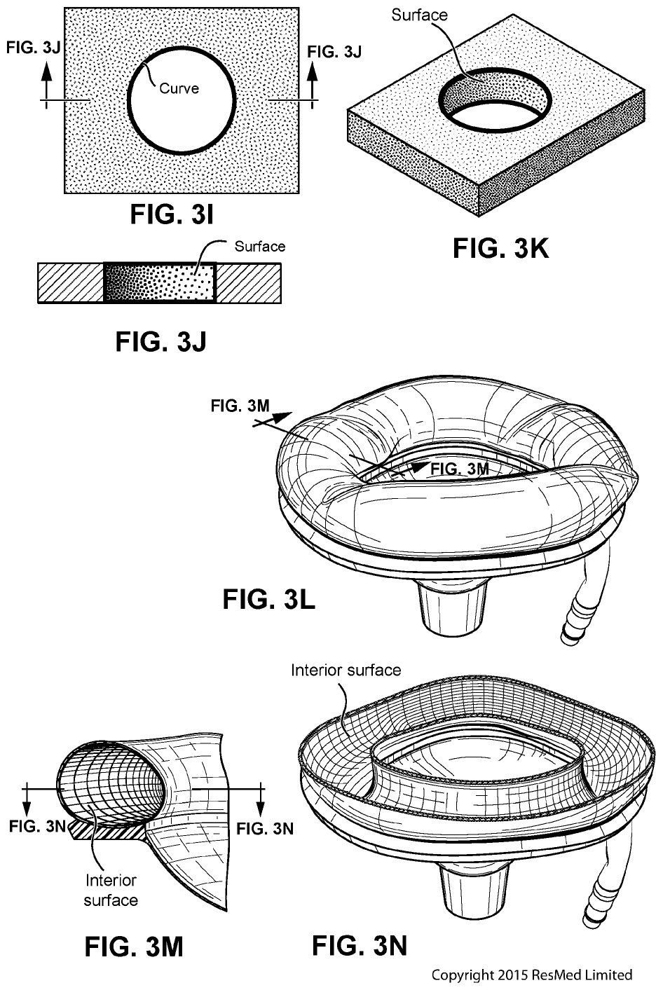

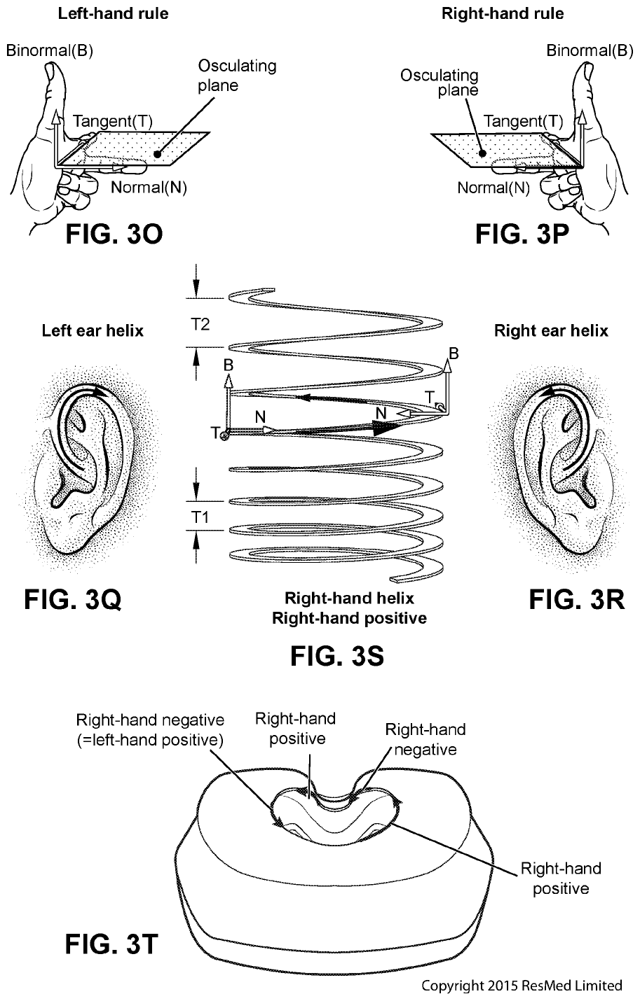

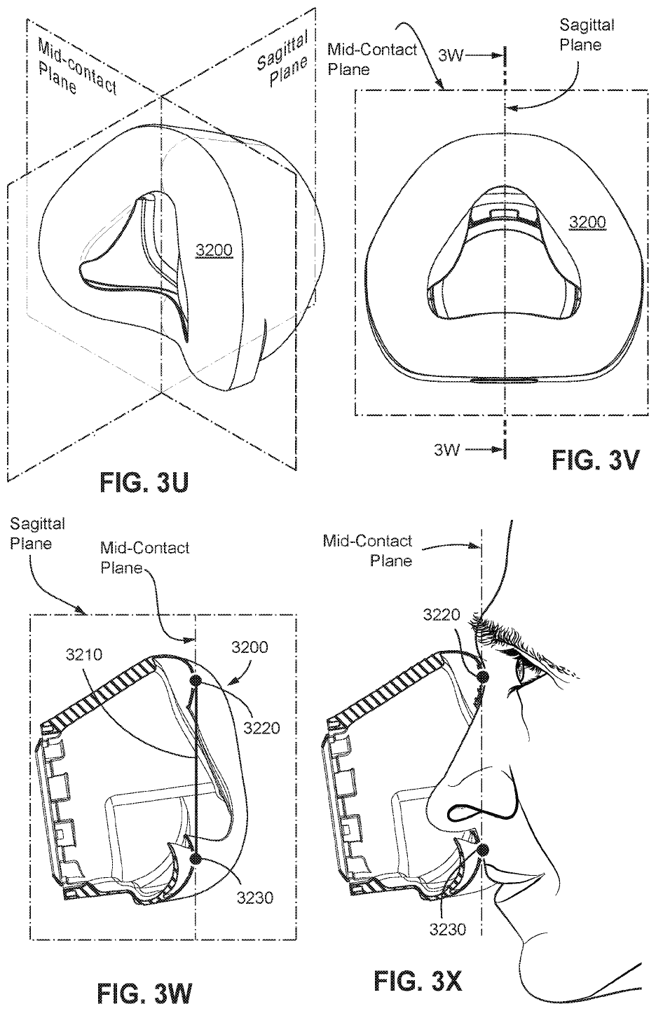

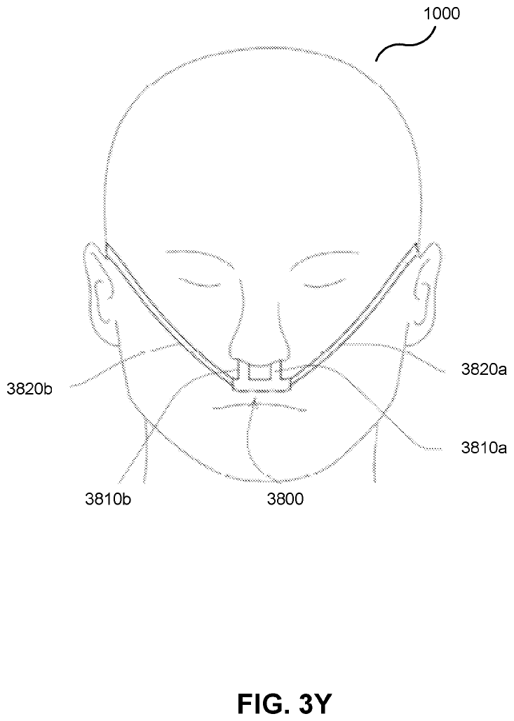

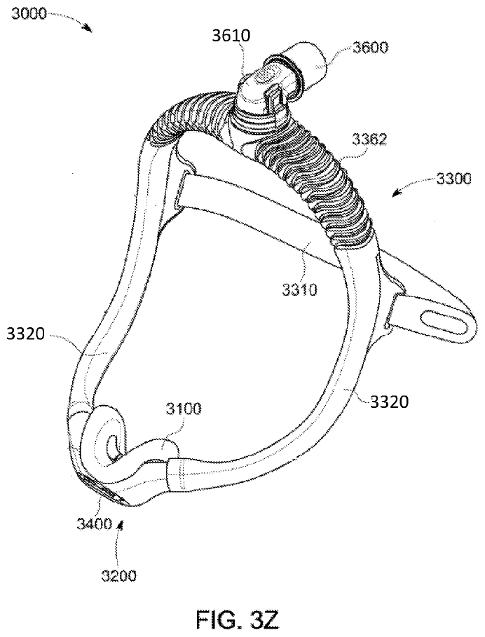

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

FIG. 3Z shows a patient interface having conduit headgear in accordance with one form of the present technology.

### 3.4 RPT Device

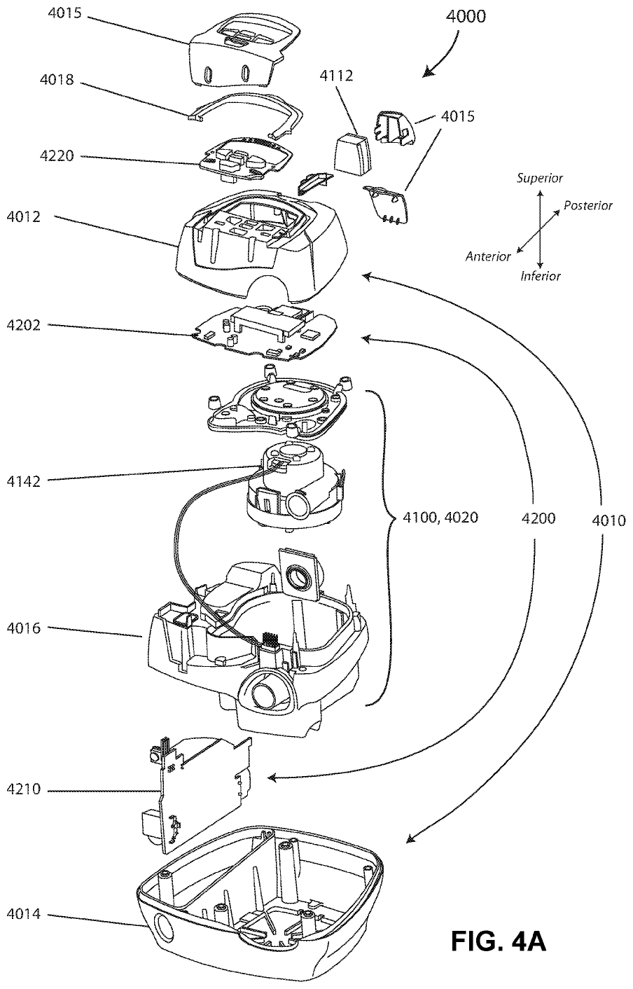

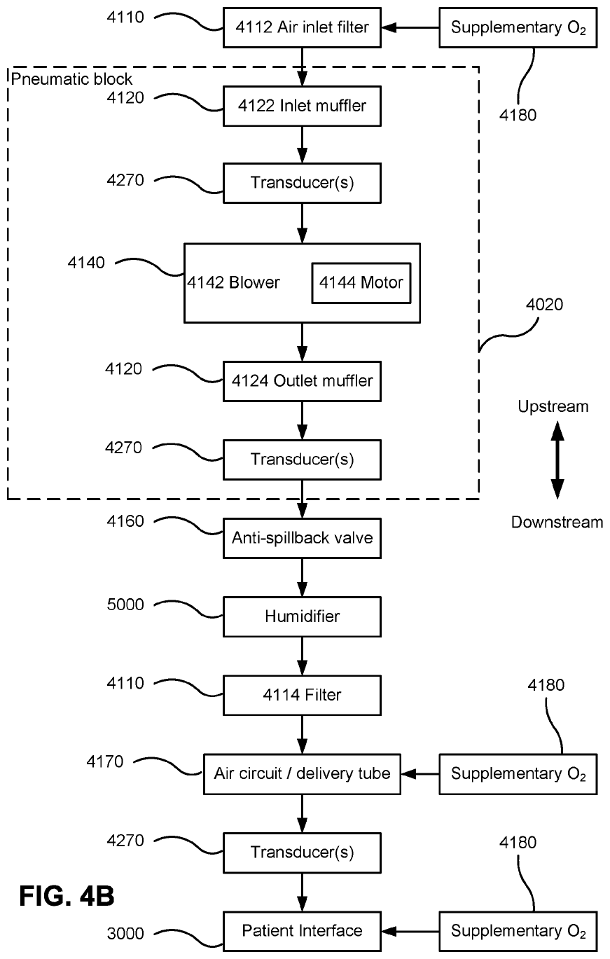

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

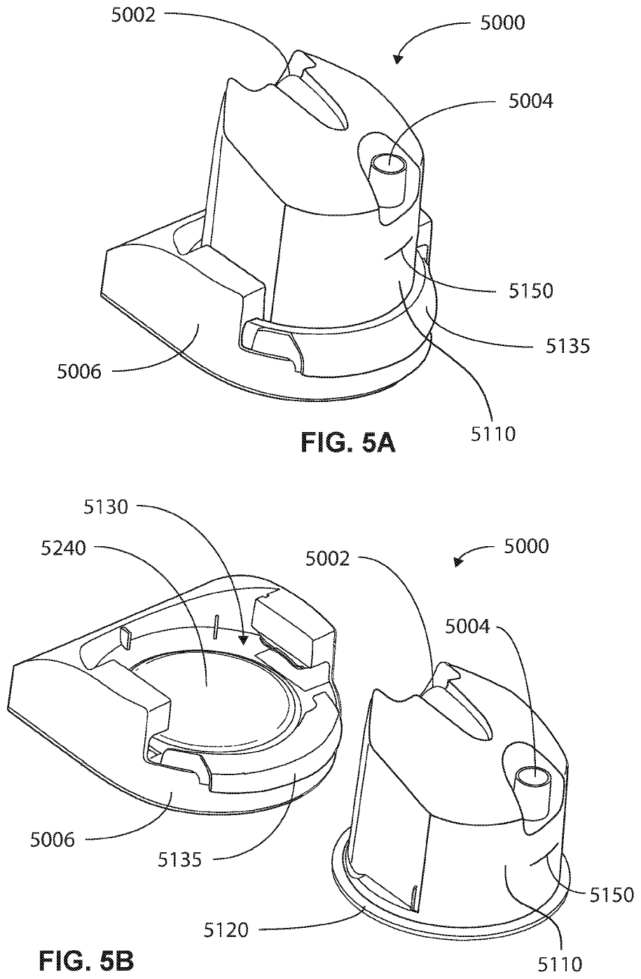

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Breathing Waveforms

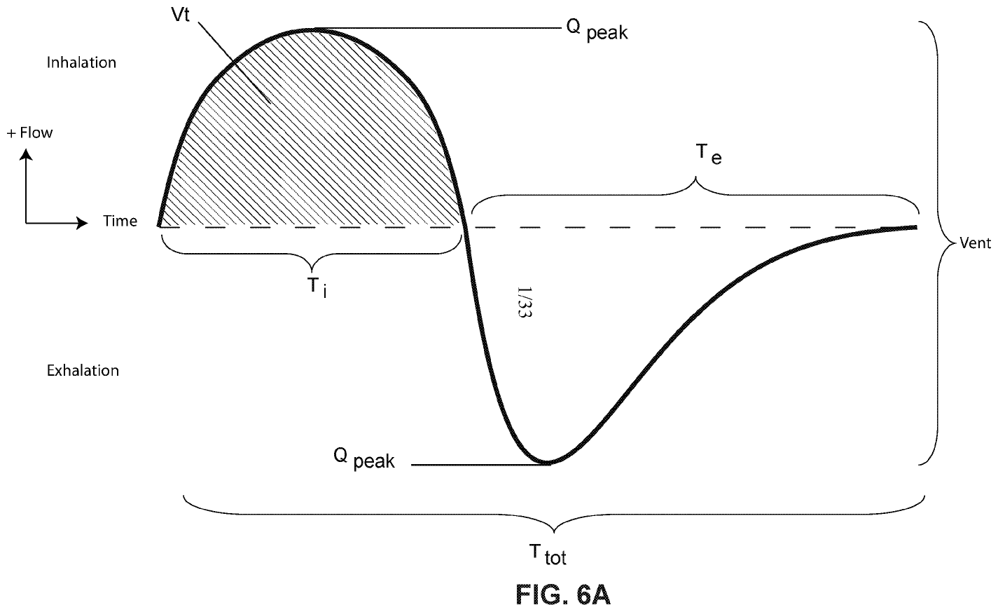

FIG. 6A shows a model typical breath waveform of a person while sleeping.

### 3.7 Patient Interfaces and Methods of Manufacture of the Present Technology

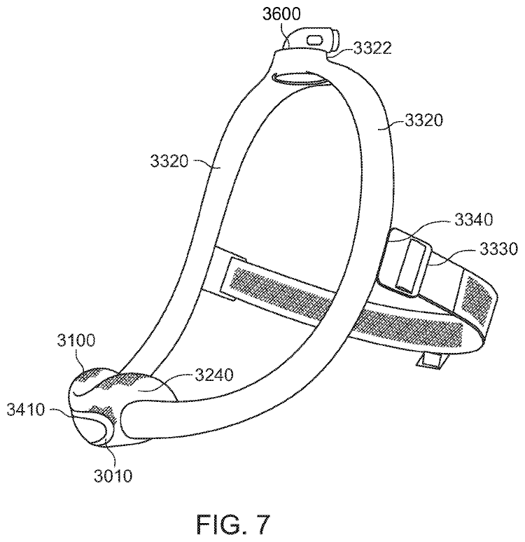

FIG. 7 shows a patient interface according to one form of the technology.

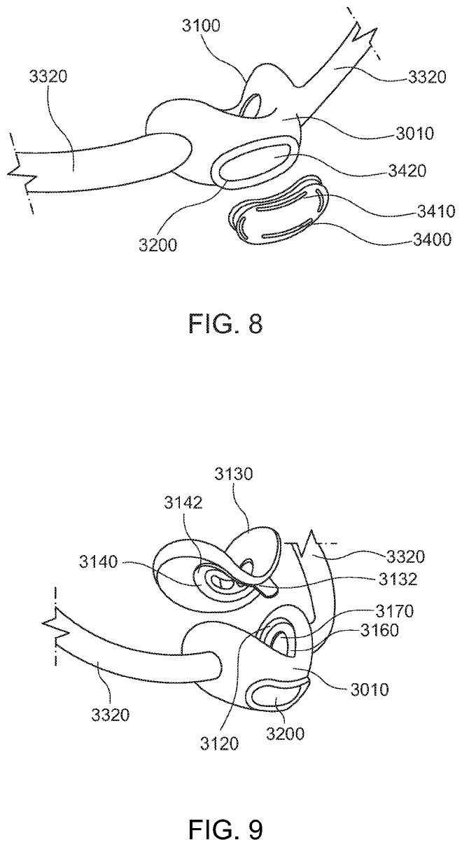

FIG. 8 shows a perspective view of a patient interface according to another form of the technology with a vent module separated from the frame.

FIG. 9 shows a perspective view of a patient interface according to one form of the technology with a seal forming structure separated from the frame.

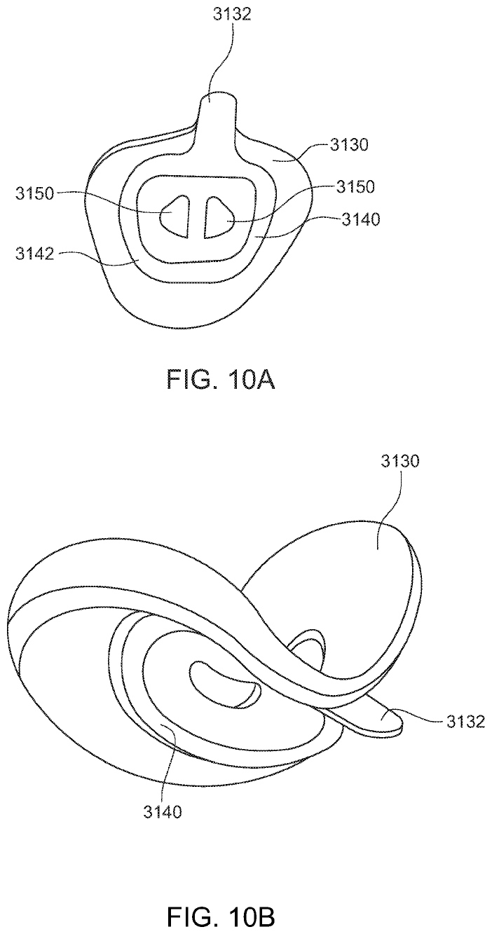

FIG. 10A shows one form of seal forming structure in an undeformed configuration.

FIG. 10B shows the seal forming structure of FIG. 10A deformed into an in-use configuration.

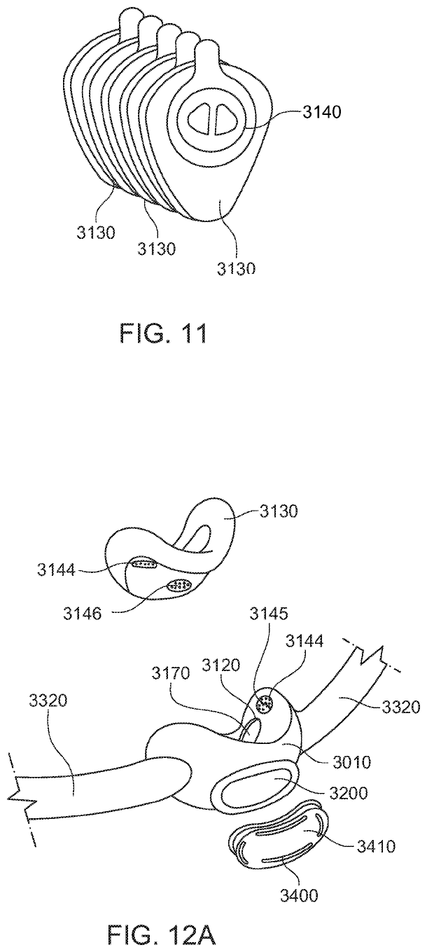

FIG. 11 shows a plurality of seal forming structures as shown in FIG. 10A nested together.

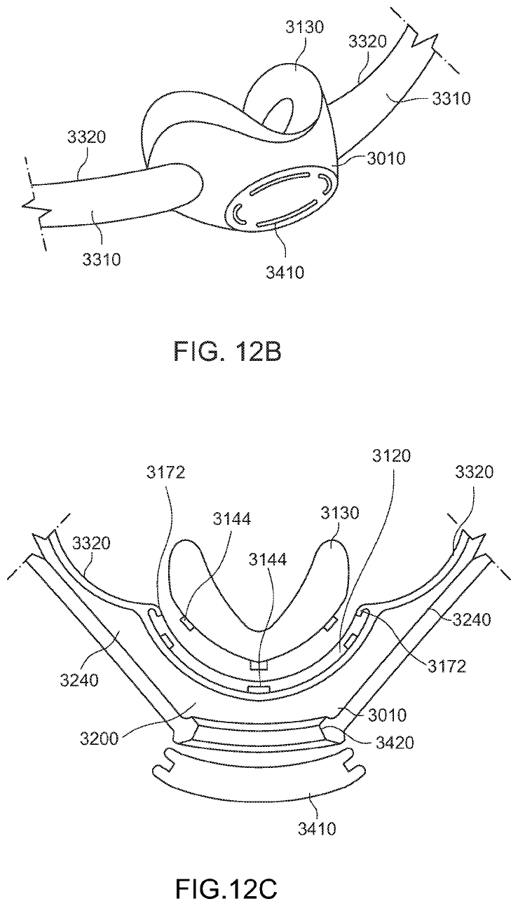

FIG. 12A is a perspective view of a patient interface according to another form of the technology with a vent module and seal forming structure separated from the frame.

FIG. 12B shows the patient interface of FIG. 12A.

FIG. 12C shows a cross-section through the patient interface of FIG. 12A.

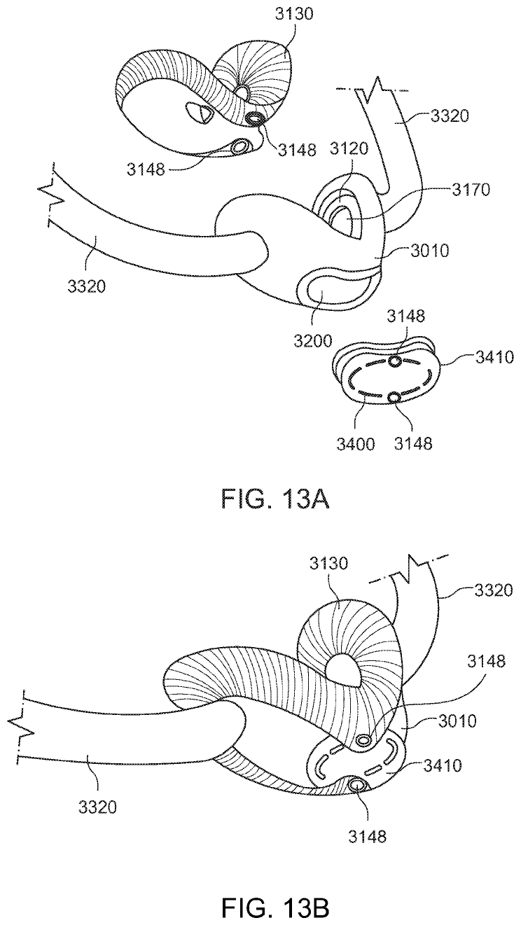

FIG. 13A is a perspective view of a patient interface according to another form of the technology with a vent module and seal forming structure separated from the frame.

FIG. 13B shows the patient interface of FIG. 13A.

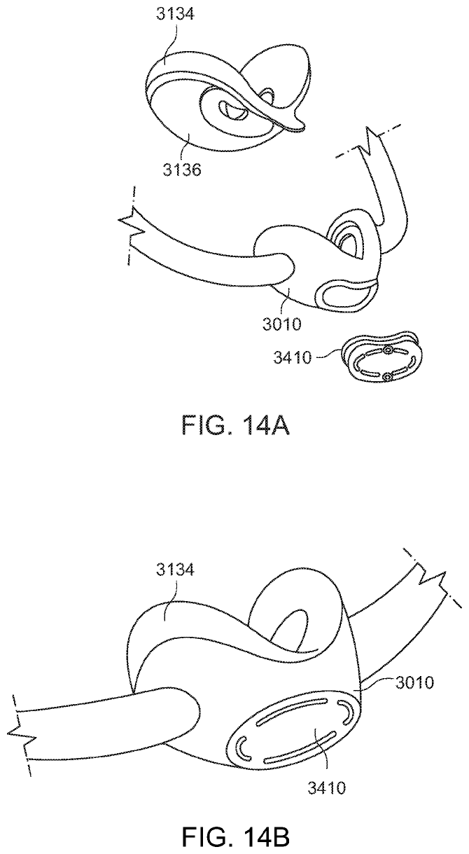

FIG. 14A shows a perspective view of a patient interface according to one form of the technology with the vent module and seal forming structure separated from the frame.

FIG. 14 B shows the patient interface of FIG. 14A.

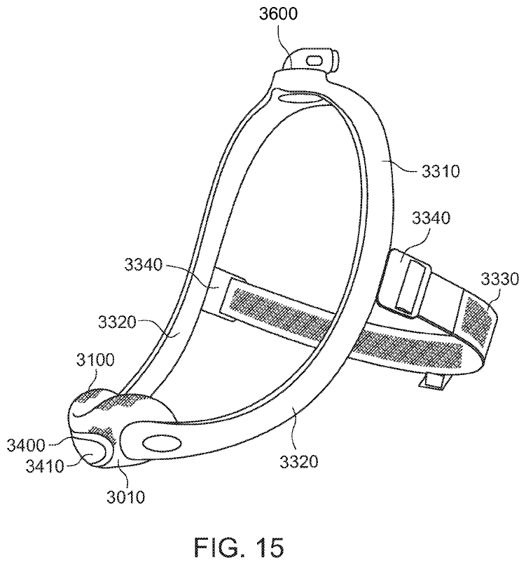

FIG. 15 is a perspective view of a patient interface according to another form of the technology.

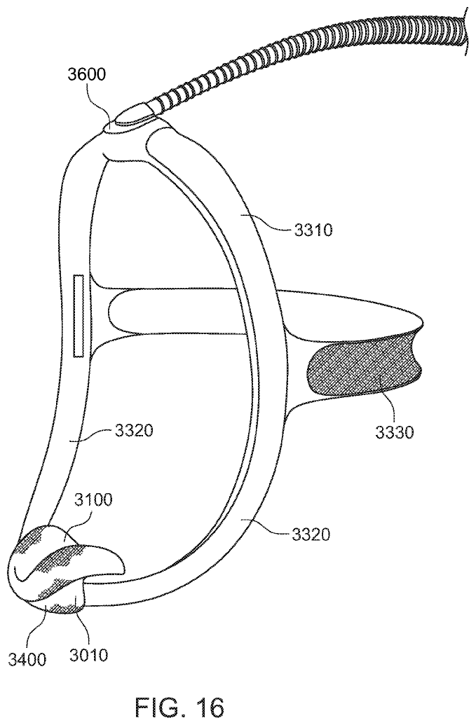

FIG. 16 is a perspective view of a patient interface according to another form of the technology.

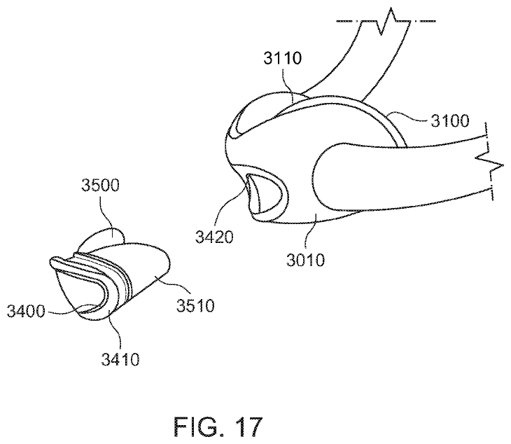

FIG. 17 is an exploded view of a patient interface according to another form of the technology.

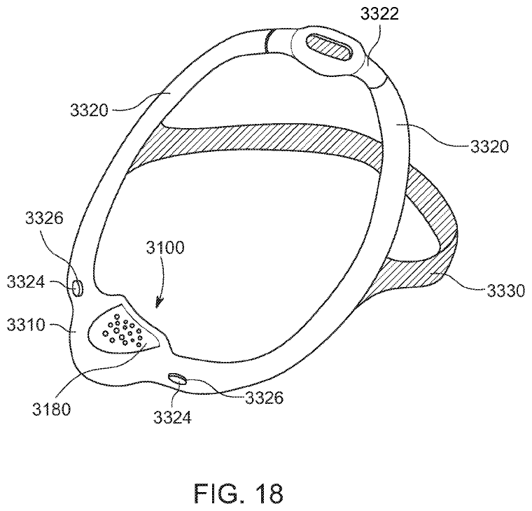

FIG. 18 is a perspective view of a patient interface according another form of the technology.

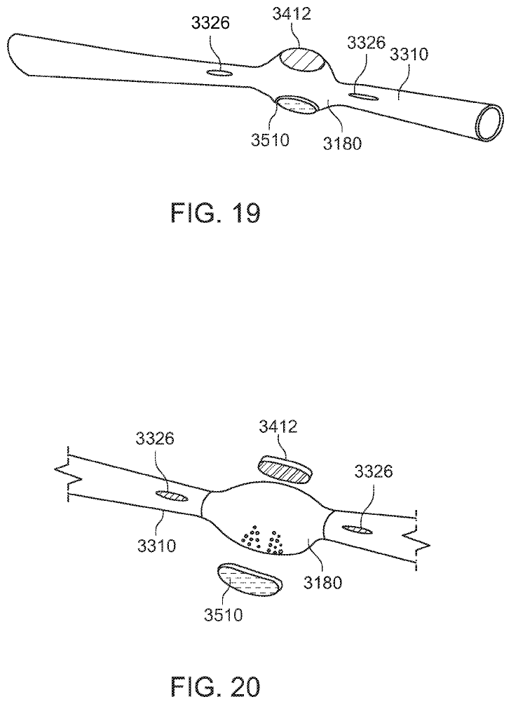

FIG. 19 shows the flexible cover of the patient interface of FIG. 18, turned inside out.

FIG. 20 shows an exploded view of the flexible cover of the patient interface of FIG. 18, turned inside out.

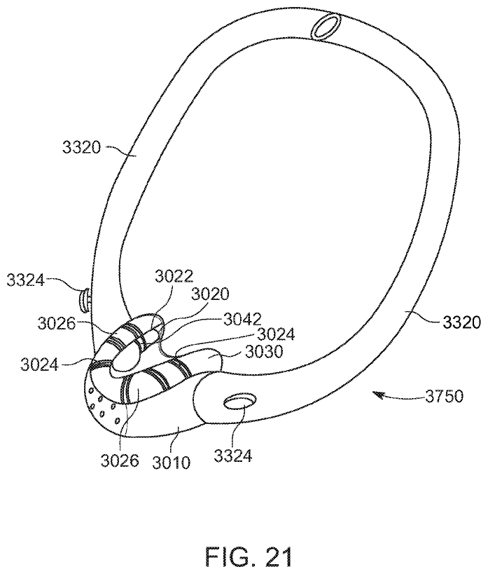

FIG. 21 is a perspective view of the substructure of the patient interface of FIG. 18.

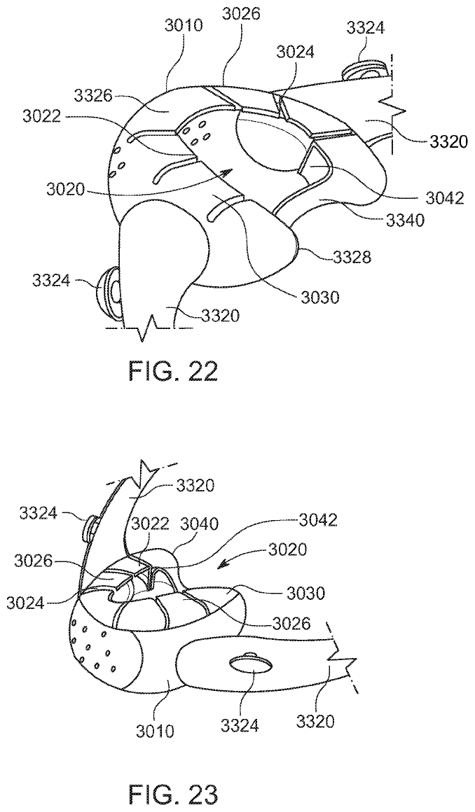

FIG. 22 is an enlarged rear perspective view of the frame of the substructure of FIG. 21.

FIG. 23 is an enlarged front perspective view of the frame of the substructure of FIG. 21.

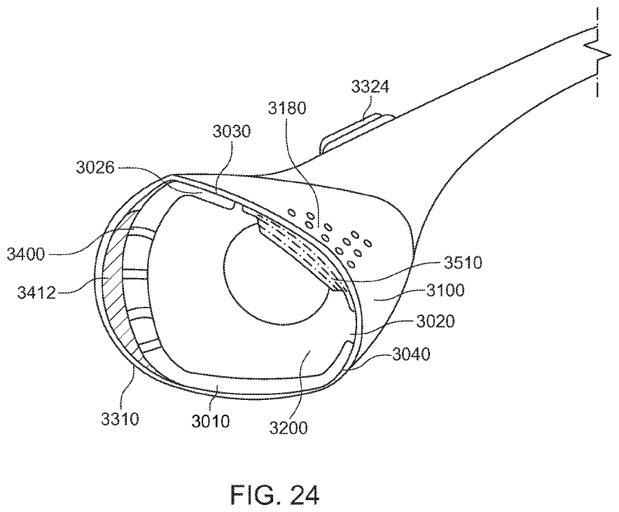

FIG. 24 is a cross-section view of the patient interface of FIG. 18.

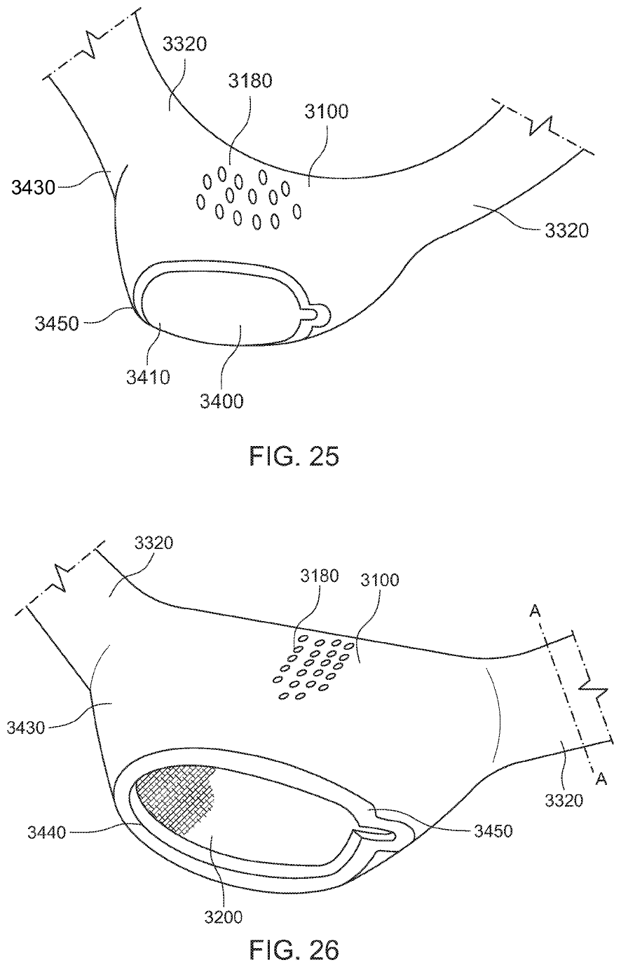

FIG. 25 is a perspective view of a patient interface according to another form of the technology.

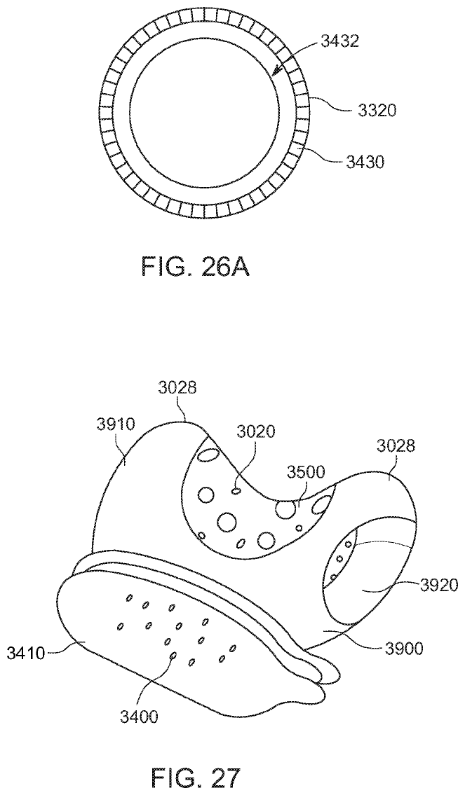

FIG. 26 is a perspective view of the patient interface of FIG. 25 with a vent module removed.

FIG. 26A is a cross-section a tube portion of the patient interface of FIG. 25.

FIG. 27 is a perspective view of an undercushion and vent module of the patient interface of FIG. 25.

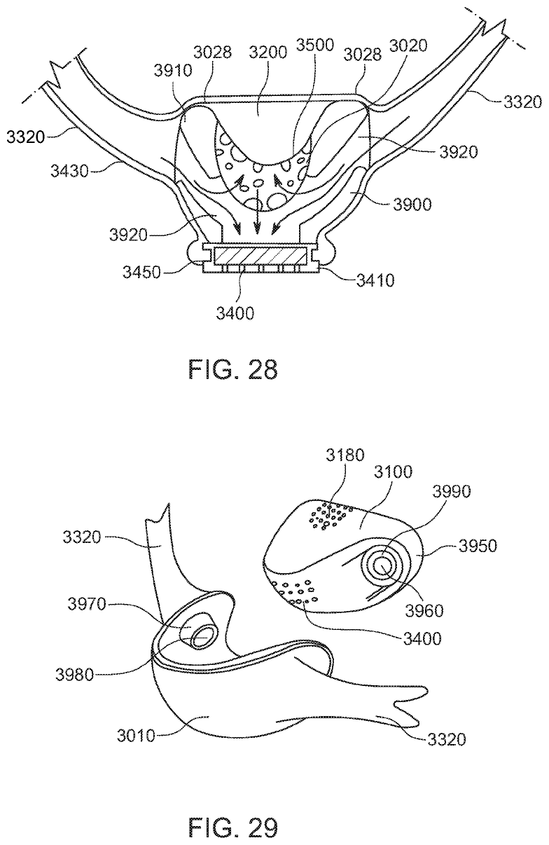

FIG. 28 is a cross-section through the patient interface of FIG. 25.

FIG. 29 is a front perspective exploded view of a patient interface according to another form of the technology.

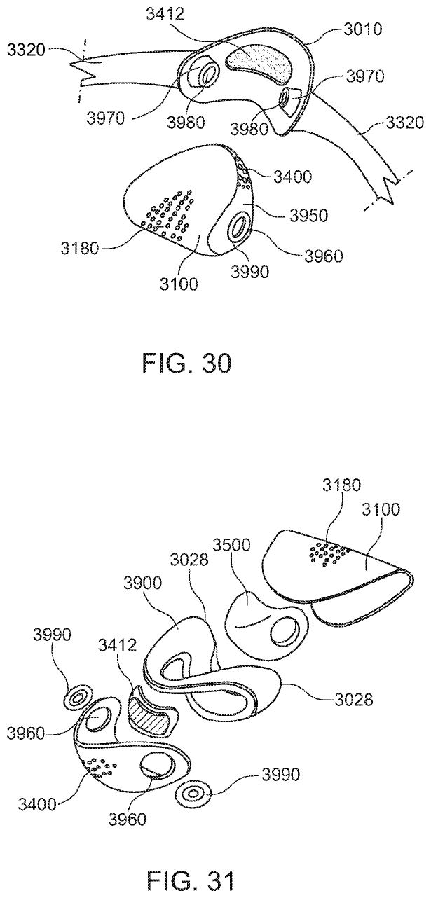

FIG. 30 is a rear perspective exploded view of the patient interface of FIG. 29.

FIG. 31 is a front perspective exploded view of the cushion module of the patient interface of FIG. 29.

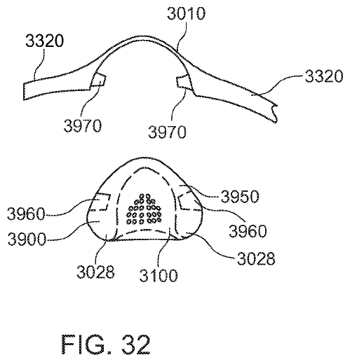

FIG. 32 is a plan exploded view of the patient interface of FIG. 29, with the undercushion and HME shown in hidden detail.

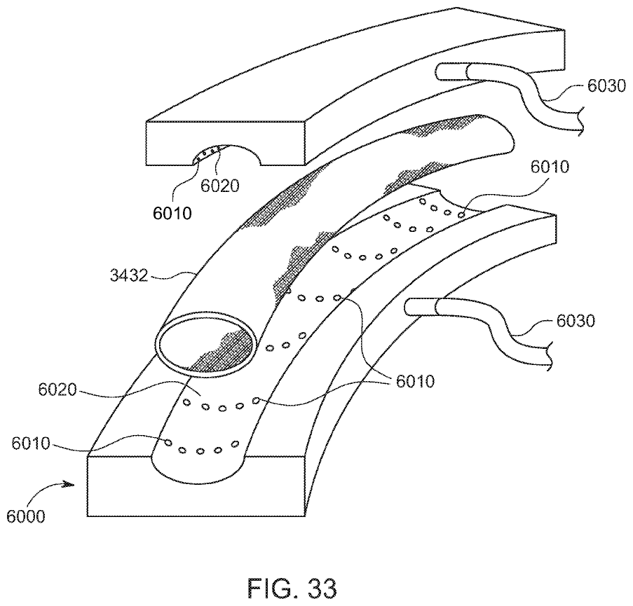

FIG. 33 is a diagrammatic exploded perspective view of a textile blank in a two-piece mould.

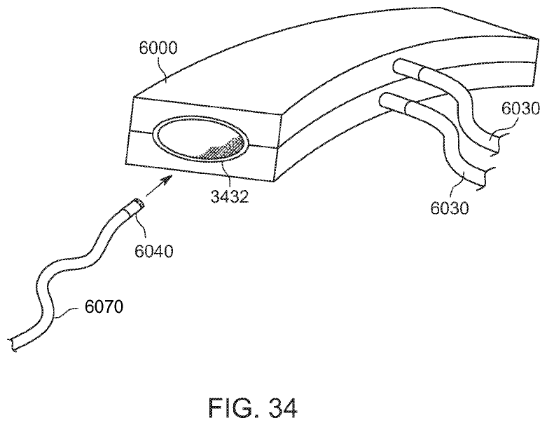

FIG. 34 is a diagrammatic perspective view of the two-piece mould and a spray nozzle.

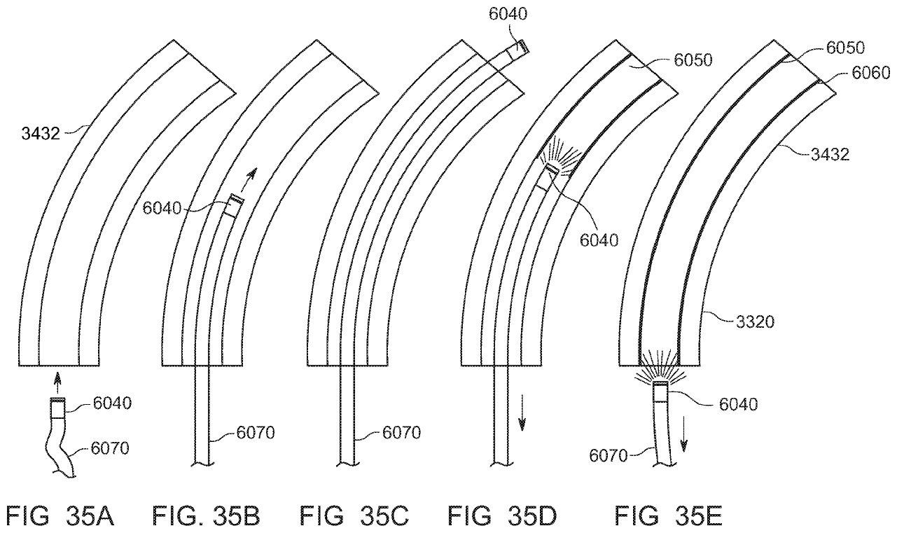

FIGS. 35A-35E show a diagrammatic cross-section view of the spray nozzle moving through the blank.

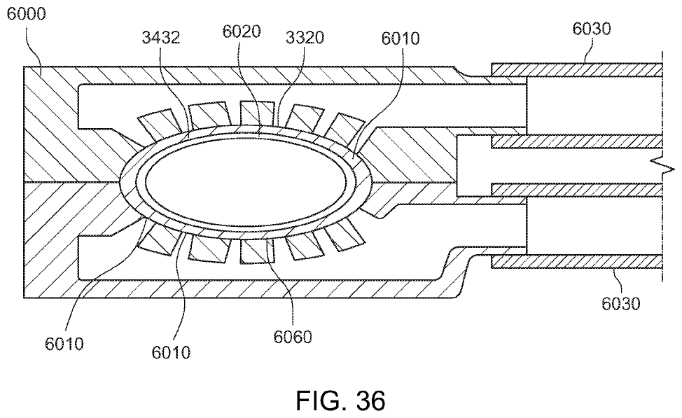

FIG. 36 is a diagrammatic transverse cross-section view of the coated blank in the mould.

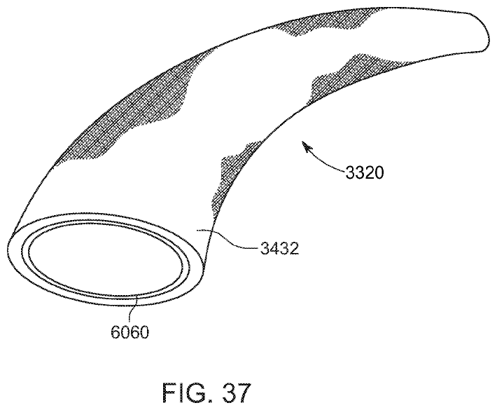

FIG. 37 shows the finished component from the method illustrated in FIGS. 33-36.

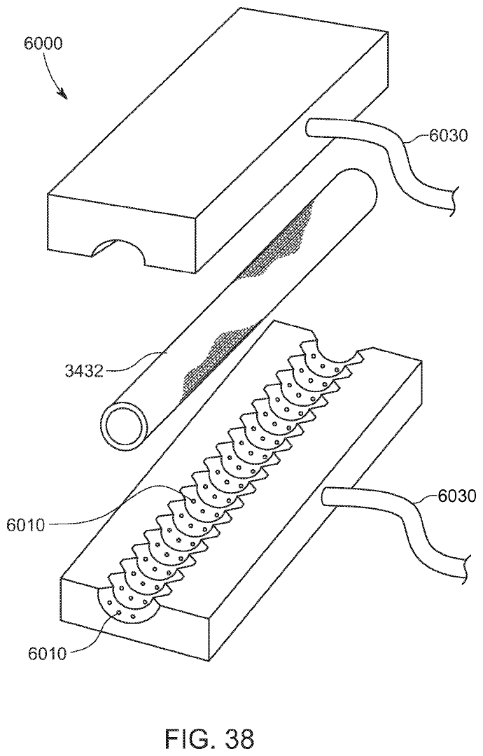

FIG. 38 is a diagrammatic exploded perspective view of a textile blank in another two-piece mould.

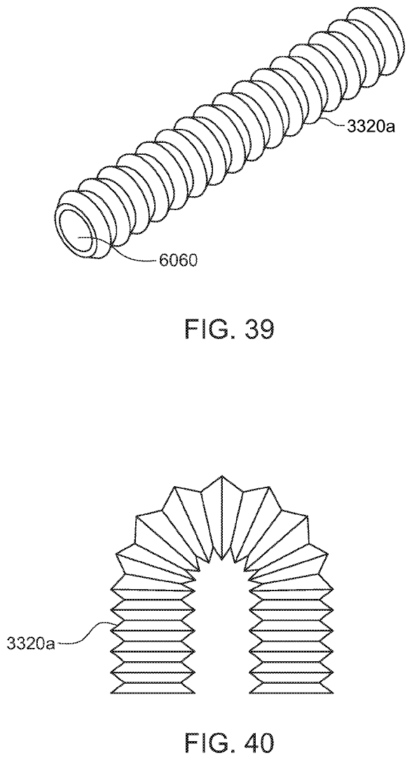

FIG. 39 shows the finished component from the mould shown in FIG. 38.

FIG. 40 shows the component of FIG. 39 in a flexed configuration.

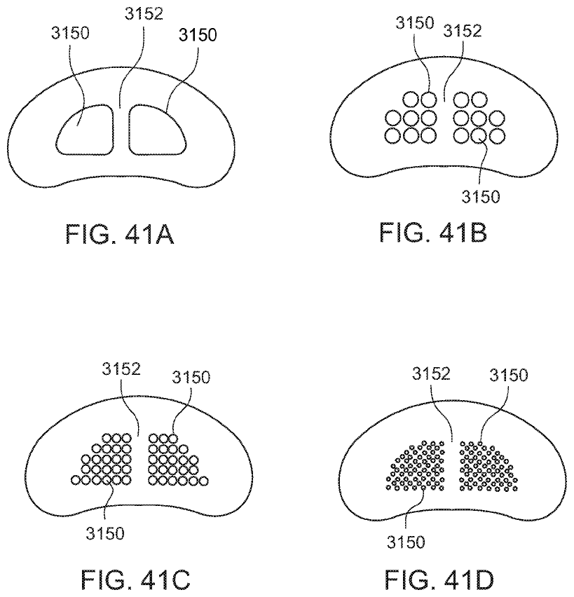

FIGS. 41A to 41D show a seal forming structure with varying numbers of holes.

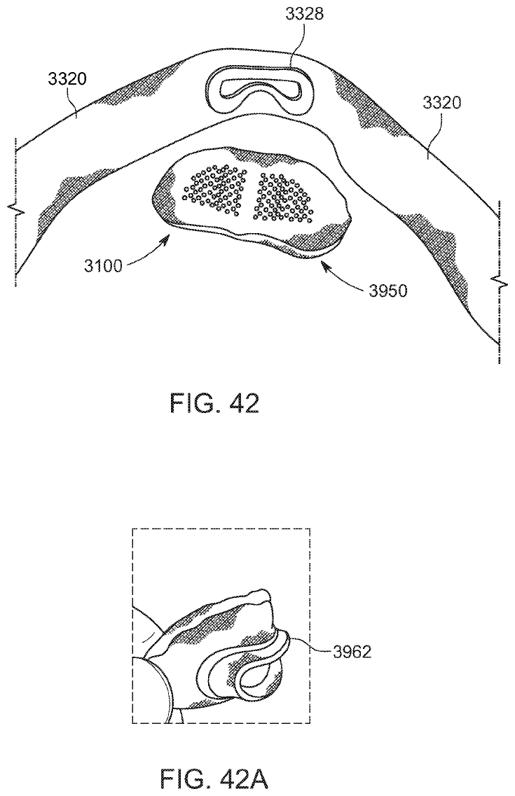

FIG. 42 shows a cushion module separated from two integrally formed tubes according to one form of the technology.

FIG. 42A shows a view of the non-patient facing side of the cushion module of FIG. 42.

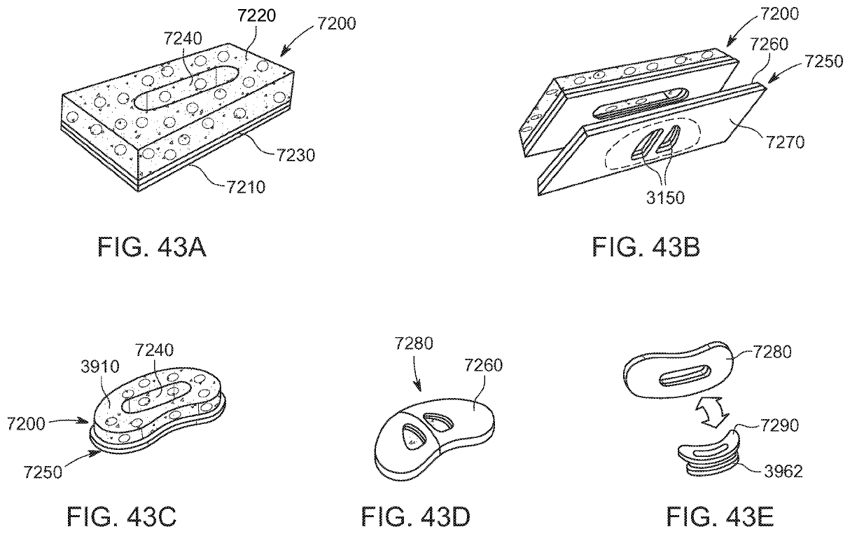

FIGS. 43A to 43E show steps in a method of manufacturing a cushion module according to one form of the technology.

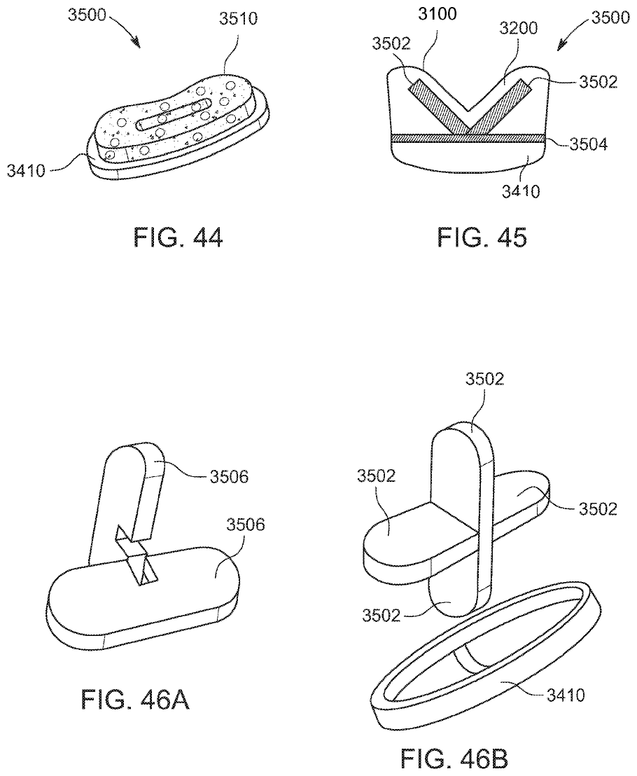

FIG. 44 shows one form of HME connected to a vent module.

FIG. 45 shows another form of HME connected to a vent module.

FIGS. 46A and 46B show steps in a method of manufacturing the HME of FIG. 45.

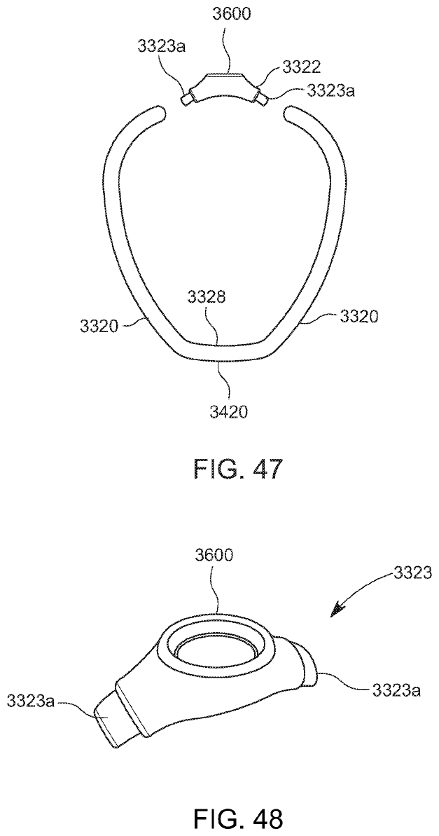

FIG. 47 shows an exploded view of a positioning and stabilising structure comprising two integrally formed tubes and a manifold portion.

FIG. 48 is a perspective view of the manifold portion of FIG. 47.

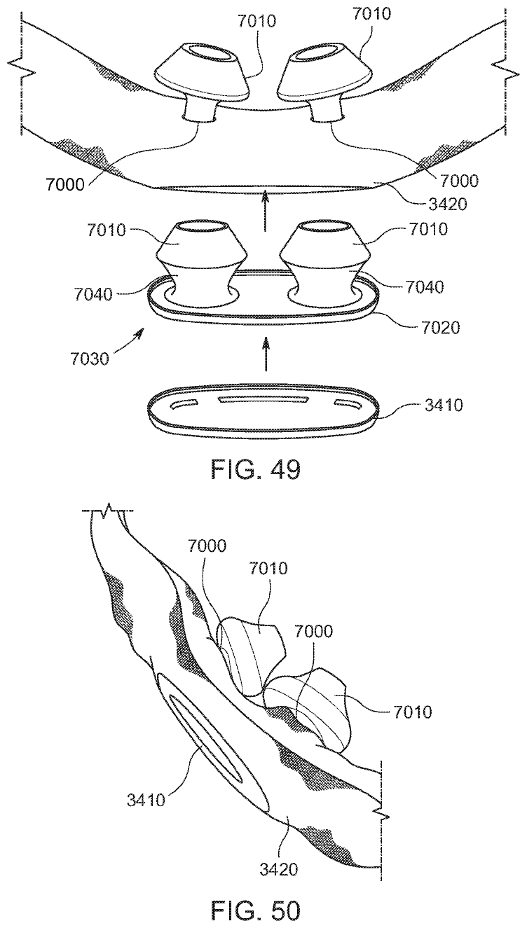

FIG. 49 is a diagram showing a method of assembly of a pillow prongs type interface according to one form of the technology.

FIG. 50 is a perspective view of the pillow prongs type interface shown in FIG. 49.

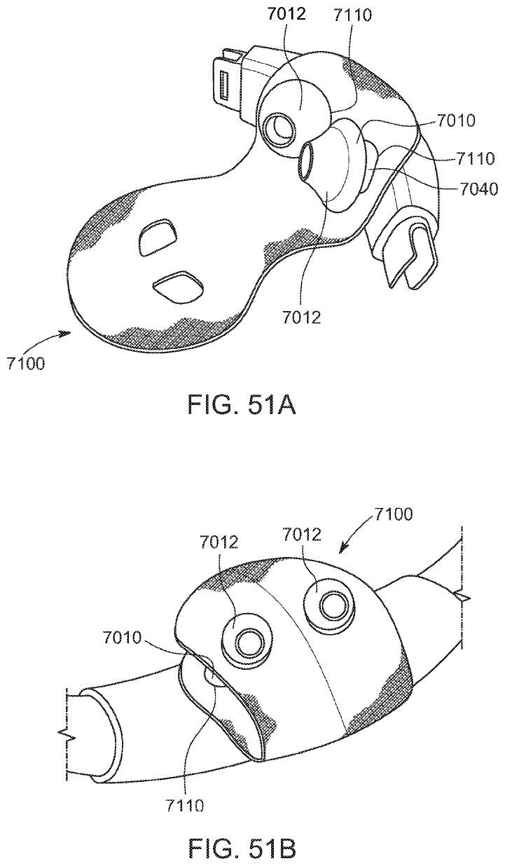

FIG. 51A is a perspective view of a textile cover partially connected to a pillow prongs type cushion module.

FIG. 51B is a different perspective view of the textile cover of FIG. 51A in a fully connected configuration.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

As shown in FIG. 3Z, a non-invasive patient interface 3000 in accordance with another aspect of the present technology comprises the following functional aspects: a seal-forming structure 3000, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 4 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs- the actual sealing surface- may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

In examples, the seal forming structure 3100 is integrally formed with the frame 3010 and plenum chamber 3200 (e.g. during the moulding process) as shown in FIGS. 7, 8, 15 16 and 17. For example a textile seal 3110 comprising a suitable impermeable backing may be formed with the frame 3010 and plenum chamber 3200, for example by overmoulding, as shown in FIG. 17, or by the method described below with reference to FIGS. 33-40.

However, in other examples, for example those shown in FIGS. 9, 12, 13 and 14, a seal support portion 3120 is integrally formed with the frame 3010 and the seal forming structure 3100 may comprise a separate component which is connectable (e.g. releasably connectable) with another component of the patient interface, e.g. the seal support portion 3120 and/or the frame 3010.

In one example, shown in FIG. 9, the seal forming structure 3100 is formed from a foam. The foam seal 3130 may be formed (e.g die cut or RF cut) from a sheet of foam and then connected to a seal support portion 3120. In examples, the foam seal 3130 may be provided with an adhesive 3140, for example adhesive tape 3142, to connect the foam seal 3130 to the seal support portion 3120. In one example, the adhesive 3140 may be applied to the foam seal 3130 in a closed curve, the curve extending around one or more apertures 3150 in the seal 3130 through which the breathable gas passes, in use, to the patient. In examples the adhesive 3140 is arranged to contact the perimeter 3160 of an aperture 3170 in the seal support portion 3120 when the foam seal 3130 is connected to the seal support portion 3120.

In one example, the foam seal 3130 may be provided with a tab 3132 which can be used to peel the foam seal 3130 away from the seal support portion 3120 when the foam seal 3130 is to be replaced. In examples where adhesive tape 3142 (or tape which has adhesive 3140 applied to it) is used to connect the foam seal 3130 to the frame 3010 and/or seal support portion 3120, the tab 3132 may be formed from the tape 3142.

As shown in FIG. 10A, the foam seal 3130 may initially be substantially flat or planar, and may be bent or deformed to conform to the shape of the seal support portion 3120. FIG. 10B shows the shape the foam seal 3130 may adopt when engaged with the frame 3010 and/or seal support portion 3120.

In examples, a plurality of different size foam seals 3130 may be configured to engage the frame 3010 and/or seal support portion 3120, such that a user may select the size of foam seal 3130 which is most comfortable for them.

As shown in FIG. 11, in the substantially flat or planar configuration a plurality of the foam seals 3130 may take up very little space when nested together. This may be particularly convenient for shipping and/or packaging of the foam seals 3130.

Referring next to FIGS. 12A-12C, in one example the seal 3130 may be connected to the frame 3010 and/or seal support portion 3120 by means of a plurality of hook and loop fastener (e.g. Velcro™) portions 3144. For example, portions of hook material 3145 may be provided to the seal support portion 3120 around an aperture 3170 in the seal support portion 3120, and portions of loop material 3146 may be provided to the corresponding portions of the foam seal 3130.

In examples, a flexible lip 3172, best seen in FIG. 12C, may be provided around the edge of aperture 3170 to form a seal between the foam seal 3130 and the seal support portion 3120.

A vent module 3410 may be provided to an aperture 3420.

Referring next to FIGS. 13A and 13B, in one example the foam seal 3130 may be connected to the seal support portion 3120 and/or frame 3010 by means of a plurality of snap fasteners 3148. In examples snap fasteners 3148 may be provided to the frame 3010 and/or to a vent module 3410 which is connectable to the frame 3010, for example by a stretch or snap fit.

Referring next to FIGS. 14A and 14B, in another example a silicone seal 3134 may be provided. In one example the silicone seal 3134 may take substantially the same shape as the foam seal shown in FIGS. 9-11. The non-patient contacting side 3136 of the silicone seal 3134 may be sticky, such that the silicone seal 3134 can be stuck to the seal support portion 3120 without the use of a separate adhesive.

In other examples, a textile seal may be substituted for any of the seals described above with reference to FIGS. 9-14.

In another form of the technology the seal forming structure 3100 may form part of a cushion module 3950 which is engageable with the tubes 3320, for example as discussed further below with reference to FIGS. 42 and 42A.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nose Only Mask

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3Z, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 4.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose and mouth mask according to the present technology is what has traditionally been identified as a “full-face mask”, having a seal-forming structure 3100 configured to seal on the patient's face around the nose, below the mouth and over the bridge of the nose. A full-face mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “full face cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

4.3.1.9 Seal Forming Structures with Multiple Holes

As is noted above, examples of the technology may comprise a nasal hole (e.g. in the seal forming structure) configured to provide air or breathable gas to the nares. However, some forms of the technology may comprise a hole 3150 for each naris, as shown in FIGS. 10A and 10B and FIG. 41A. Other forms of the technology may comprise a plurality of holes 3150 for each naris. For example, the example shown in FIG. 41B comprises 8 holes 3150 for each naris. The holes 3150 may be, for example, of about 4 mm diameter. FIG. 41C shows an example with 23 holes 3150 for each naris. The holes 3150 may be, for example, of about 2 mm diameter. FIGS. 41D and 42 show examples with 61 holes 3150 for each naris. The holes 3150 may be, for example of about 0.8 mm diameter.

In examples, the sets of holes 3150 for each naris may be grouped together, with a space or strip 3152 between each group having no holes. In use, the space or strip 3152, which is engaged by the patient's columella.

In examples, the holes 3150 may be made by die cutting. However, for very small size holes (e.g. 0.8 mm diameter) laser cutting may be used.

By providing multiple small holes 3150 for each naris, the likelihood of damaging the seal forming structure 3100, particularly during cleaning, may be reduced. In addition, providing multiple small holes for each naris may diffuse the air flow and may reduce the likelihood of “jetting”. Furthermore, sealing may be improved.

The examples shown in FIGS. 18, 24, 25 and 30 may also be provided with multiple holes for each naris.

### 4.3.1.10 Removable Pillow Prongs

Referring next to FIGS. 49 and 50, in one example an interface with two integrally formed tubes 3320 (as described further herein) may be provided with an aperture 3420 for a vent module 3410 on an anterior/inferior side of the tubes 3320 and two further prong receiving apertures 7000, provided to the opposite side of the tubes to the vent module aperture 3420, for receiving a pair of nasal pillow prongs 7010.

In one form of the technology the pillow prongs 7010 may be formed integrally with a base component 7020 to form a prongs module 7030. The prongs module 7030 may be configured to be insertable into the integrally formed tubes 3320 though the vent module aperture 3420, with each pillow prong 7010 extending through a respective one of the prong receiving apertures 7000.

Each pillow prong 7010 may comprise a reduced diameter neck portion 7040 (e.g. a groove or similar) which is engaged by the wall of the tubes 3320 when the pillow prongs 7010 are in their in-use position, to thereby hold the pillow prongs 7010 in the required position.

### 4.3.1.11 Pillow Prongs Cover

Referring next to FIGS. 51A and 51B, in one form of the technology a textile cover 7100 may be provided for a pillow prongs interface such as the one shown in FIGS. 50 and 51.

The cover 7100 may have a first pair of prong engaging apertures 7110 which are configured to engage the neck portions 7040 of a pair of pillow prongs 7010. The cover 7100 may have a second pair of apertures 7120 which are configured to allow at least a portion of the sealing face 7012 of the pillow prongs 7010 to extend through the cover 7100.

In one form of the technology, shown in FIG. 51B, the textile cover 7100 may be configured to extend on the inferior/posterior side of the pillow prongs 7010, from the neck 7040 of the pillow prongs 7010 to the sealing surface 7012 of the pillow prongs. Such an example may provide a soft surface for contact with the patient's top lip, in use.

In another form of the technology (not shown) a larger textile cover may be configured to extend from the necks 7040 of the pillow prongs 7010, around the circumference of the tubes 3020 (e.g. over the vent or vent module) to the sealing surface 7012 of the pillow prongs. Such an example may also provide a soft surface for the patient's top lip, but may have the additional function of diffusing vent flow from the vent module.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 4.3.3 Positioning and Stabilising Structure

The seal-forming structure 3100 of the patient interface 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 4.3.3.1 Conduit Headgear

In examples of the technology the positioning and stabilising structure 3300 comprises at least one (e.g. two) tubes 3320 which are configured to extend from a port 3600 positioned, in use, on top of the patient's head (or from a manifold comprising a port) to the frame 3010. The tubes 3320 allow flow from the port 3600 to inlet ports 3240 in the plenum chamber 3200 (e.g. “conduit headgear”).

In some forms of the present technology, the positioning and stabilising structure 3300 comprises one or more headgear tubes 3320 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3320 that deliver air to the plenum chamber 3200 from the air circuit 4170. The tubes 3320 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head. The patient interface 3000 shown in FIGS. 7-32 also includes gas delivery tubes 3320 forming a conduit headgear. The patient interface shown in FIG. 42 has two integrally formed gas delivery tubes 3320 which form a conduit headgear, as described further herein.

In the form of the present technology illustrated in FIG. 3Z, the positioning and stabilising structure 3300 comprises two tubes 3320, each tube 3320 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3320 is compressed to block or partially block the flow of gas along the tube 3320, the other tube 3320 remains open to supply pressurised gas to the patient. In other examples of the technology, the patient interface 3000 may comprise a different number of tubes, for example one tube, or two or more tubes. In one example in which the patient interface has one tube 3320, the single tube 3320 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head.

In the form of the technology shown in FIG. 3Z the two tubes 3320 are fluidly connected at superior ends to each other and to the connection port 3600. In some examples, the two tubes 3320 are integrally formed while in other examples the tubes 3320 are formed separately but are connected in use and may be disconnected, for example for cleaning or storage. Where separate tubes are used they may be indirectly connected together, for example each may be connected to a T-shaped connector having two arms/branches each fluidly connectable to a respective one of the tubes 3320 and a third arm or opening in the T-shaped connector providing the connection port 3600 for fluid connection to the air circuit 4170 in use.

The tubes 3320 may be formed from a flexible material, such as an elastomer, e.g. silicone or TPE, and/or from one or more textile and/or foam materials. The tubes 3320 may have a preformed shape and may be able to be bent or moved into another shape upon application of a force but may return to the original preformed shape in the absence of said force. The tubes 3320 may be generally arcuate or curved in a shape approximating the contours of a patient's head between the top of the head and the nasal or oral region.

In some examples, the one or more tubes 3320 are crush resistant to resist being blocked if crushed during use, for example if squashed between a patient's head and pillow, especially if there is only one tube 3320. The tubes 3320 may be formed with a sufficient structural stiffness to resist crushing or may be as described in U.S. Pat. No. 6,044,844, the contents of which are incorporated herein by reference.

Each tube 3320 may be configured to receive a flow of air from the connection port 3600 on top of the patient's head and to deliver the flow of air to the seal-forming structure 3100 at the entrance of the patient's airways. In the example shown in FIG. 3Z, each tube 3320 lies in use on a path extending from the plenum chamber 3200 across the patient's cheek region and superior to the patient's ear to the elbow 3610. For example, a portion of each tube 3320 proximate the plenum chamber 3200 may overlie a maxilla region of the patient's head in use. Another portion of each tube 3320 may overlie a region of the patient's head superior to an otobasion superior of the patient's head. Each of the tubes 3320 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3320 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3320, may be structured and arranged to pull the seal-forming structure 3100 into the patient's face under the nose with a sealing force vector in a posterior and superior direction (e.g. a posterosuperior direction). A sealing force vector with a posterosuperior direction may cause the seal-forming structure 3100 to form a good seal to both the inferior periphery of the patient's nose and the anterior-facing surfaces of the patient's face, for example, on either side of the patient's nose and the patient's lip superior.

In some examples of the present technology, one or both of the tubes 3320 are not extendable in length. However, in some forms, the tubes 3320 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3Z comprises tubes 3320, the superior portions of which comprise extendable tube sections each in the form of an extendable concertina structure 3362.

In one form of the technology, shown in FIG. 47, the two tubes 3320 may be integrally formed, e.g. manufactured as one piece. The tubes 3320 may be connected, in use, to a manifold component 3323 which comprises a port 3600. The manifold component 3323 may comprise rigid or semi-rigid connector portions 3323a for connecting the manifold portion 3323 to the tubes 3320. In examples, the manifold component 3323 may be made from a relatively soft silicone and the connector portions 3323a may be formed by overmoulding with a harder material.

In some forms of the technology, no part of the integrally formed tubes 3320 has a Shore A hardness of more than 60, or more preferably, no part has a Shore A hardness of more than 40. In examples the tubes 3320 may be formed from silicone. In other examples, the tubes 3320 may comprise a textile, as described further herein. The integrally formed tubes 3320 may be provided with an aperture 3328 to allow connection to a cushion module, for example as shown in FIG. 42, and an aperture 3420 for a vent module.

Manufacturing the tubes 3320 as one integral component may eliminate the presence of hard connector parts near the patient's face (other than the connector 3962 on the anterior side of the cushion module 3950). In particular, there may be no hard connectors positioned to make contact with the side of the patient's face if they are sleeping on their side.

Furthermore, the integral tube components 3320 can be manufactured by a moulding process which uses two sliding cores (not shown) which meet at the intersection of the tubes 3320. During demoulding, these cores can be removed in parallel. This may simplify the moulding process when compared to some processes for moulding tubes of the prior art.

### 4.3.4 Flexible Cover

As shown in FIGS. 15 and 16, in examples a flexible cover 3310 is provided over at least a portion of each tube 3320. The flexible cover 3310 may be provided for at least portions of each tube 3320 which would otherwise contact the patient's face.

In examples, a single flexible cover 3310 covers portions of each tube 3320, the frame 3010 and optionally at least a portion of a seal support portion 3120. In some forms of the technology, the flexible cover 3310 comprises a seal forming portion which forms a seal between the patient's airways and the plenum chamber 3200.

In some examples, the seal forming portion 3180 may comprise a textile with a silicone backing layer. However, in other examples no backing layer may be required.

In examples, the flexible cover 3310 comprises a textile, for example a knitted textile. Circular knitting or 3D knitting may be used to form the flexible cover 3310.

In some forms of the technology, the flexible cover 3310 is provided with openings or “windows” to allow the patient to inspect the cleanliness of the tubes.

In some forms of the technology, the flexible cover 3310 may comprise an integrally formed back strap 3330, as shown in FIG. 16. In other forms of the technology a textile back strap 3330 may be formed separately from the cover 3310 and may be connected to the cover 3310, for example by stitching, welding or adhesive. In other forms of the technology the flexible cover 3310 may be provided with at least one strap connecting structure, for example tabs 3340, to allow connection of a back strap 3330 to the cover 3310, as shown in FIG. 15. Alternatively, the strap connecting structures such as tabs 3340 may be formed integrally with the tubes 3320, as shown in FIG. 7.

### 4.3.5 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

In one form, a vent module 3410 is provided which comprises at least one vent. The vent module 3410 may be formed separately from the frame 3010. The vent module 3410 may be engageable with the frame 3010 (for example, removably engageable) for example by means of a snap or stretch fit, as shown in FIGS. 9, 12, 13, 14 and 17. In other examples, a vent module 3410 may be engaged with the frame 3010 by alternative means, for example welding or overmoulding. A vent module 3410 may also be engageable with one or more gas delivery tubes 3320, as described below with reference to FIGS. 47, 49 and 50.

In examples of the technology in which a flexible cover partially or completely covers or overlies a vent, a diffuser material may be connected to or integrally formed with the flexible cover, as shown in FIGS. 19, 20 and 24.

### 4.3.6 Heat and Moisture Exchanger (HME)

In examples, a heat and moisture exchanger (HME) 3500 may be provided within the plenum chamber 3200. In one example, the HME 3500 is connected to the vent module 3410, for example as shown in FIG. 17, to form a discrete component. In the example in FIG. 17 the vent module 3410 is connected to the frame 3010 by a stretch fit, so the vent module 3410 and HME 3500 can easily be removed from the frame 3010 and replaced and/or the interior of the plenum chamber 3200 can be inspected and/or cleaned.

The HME 3500 may comprise open cell reticulated polyester foam HME material 3510. The foam may be salted (e.g. impregnated with a salt such as calcium chloride) or unsalted. Impregnating the foam with a salt may increase its ability to absorb and desorb moisture. However, salt impregnated foam may become less effective if washed, and so non-impregnated foam may be preferred in examples in which washing of the HME 3500 is intended.

In examples, the HME 3500 is configured to substantially fill the plenum chamber 3200 such that little or none of the interior volume of the plenum chamber 3200 is not filled with HME 3500 material. In some examples the HME 3500 may fill more than 90% of the volume of the plenum chamber 3200. The HME material 3510 may be formed to a shape which is complementary to that of the plenum chamber 3200.

In examples, the HME 3500 is removable from the plenum chamber 3200 so that the patient can inspect the interior of the plenum chamber 3200 for cleanliness.

As best seen in FIG. 44, in one example the HME 3500 may comprise a roll of foam HME material 3510, one end of which is connected to the vent module 3410.

In another form of the technology, the HME 3500 may comprise two arm portions 3502 which extend into the plenum chamber 3200, and a base portion 3504 which extends across the vent module 3410.

As shown in FIG. 46A, the HME 3500 may comprise two portions 3506 of HME material 3510 which are slotted so as to allow them to be connected in a cruciform shape, as shown in FIG. 46B. Two arms 3502 of the cruciform shape may be connected to the vent module 3410 to form the base portion 3504. The base portion 3504 may extend across the entire vent module 3410 such that any flow through the vent 3400 must pass through the HME material 3510. The two other arms 3502 of the cruciform shape may extend into the plenum chamber, as shown in FIG. 45.

An HME 3500 may be provided to any of the examples of the technology shown in FIGS. 7-32.

### 4.3.7 Decoupling Structure(s)

In one form the patient interface 3000 includes at least one decoupling structure, for example, a swivel or a ball and socket.

### 4.3.8 Connection Port

Connection port 3600 allows for connection to the air circuit 4170.

### 4.3.9 Forehead Support

In one form, the patient interface 3000 includes a forehead support 3700.

### 4.3.10 Anti-Asphyxia Valve

In one form, the patient interface 3000 includes an anti-asphyxia valve.

### 4.3.11 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

4.3.12 Patient Interface with Integrally Formed Components

Referring next to FIG. 7, in one form of the technology, a patient interface comprises a frame 3010 which defines, at least in part, the plenum chamber 3200, and a pair of tubes 3320 seamlessly connected to the frame 3010 which both supply pressurised air to the plenum chamber 3200 and which form, at least in part, a positioning and stabilising structure 3300. In the embodiment shown in FIG. 7 a manifold portion 3322 comprising a connection port 3600 is also formed integrally with the tubes 3320 and frame 3010.

In examples of the technology, the frame 3010 and tubes 3320 are integrally formed, e.g. are formed as a unitary structure that is created as one piece or a single unit. The frame 3010 and tubes 3320 are integrally formed, for example, by injection moulding or other suitable techniques. The phrase “integrally formed” is used to refer to parts (or components) of the patient interface that are formed as a unitary structure having no seams between the parts/components of the patient interface. For example, integrally formed parts (e.g. portions) may be moulded or formed together in the same mould cavity (e.g. in a single shot) or formed as one piece by an additive manufacturing (e.g. 3D printing) process.

In examples a seal forming structure 3100 or a seal support structure 3120 are also integrally formed with the frame 3010 and tubes 3320. Strap connecting structures such as tabs 3340 may be formed integrally with the tubes 3320, as shown in FIG. 7. In another form of the technology a back strap may be formed integrally with the tubes 3320.

Patient interfaces in accordance with the present technology may be simple, light weight, durable, low cost and/or easy to clean, and/or may exhibit improved sealing characteristics relative to some interfaces of the prior art. In addition, patient interfaces in accordance with the present technology which are made of flexible polymer material may be easier to fold into a compact configuration, thereby allowing space for retail shelves, enabling easy postage and is travel friendly. Having multiple parts of the patient interface (e.g., the frame 3010 and tubes 3320; or the seal forming structure 3100/seal support structure 3120 with the frame 3010 and tubes 3320; or the strap connecting structures such as tabs 3340 and tubes 3320) to be integrally formed as a unitary structure, as described above, enables the unitary structure to be seamless both from a visual and tactile perspective. Further, the patient interfaces as described in accordance with the present technology are devoid of bulky or rigid connectors between the frame/plenum chamber and the tube which leads to improved seal and comfort.

In one form of the technology a seal forming structure 3100 may be formed from a textile. A suitable impermeable backing layer, for example silicone, may be attached to the textile.

In examples, a patient interface made in accordance with the technology may comprise a frame and a plurality of tubes, wherein no part of the frame or tubes are made from a material having a Shore A Durometer hardness of greater than 80, for example no more than 60, for example no more than 40.

Referring next to FIGS. 18-24, a patient interface according to another form of the technology is shown. In this example the flexible cover 3310 substantially or completely covers the frame 3010.

The patient interface comprises a frame 3010 which is integrally formed with at least one headgear tube. For example, the frame 3010 and the at least one headgear tube are formed as a unitary structure that is created as one piece or a single unit having no seams between the frame 3010 and the at least one headgear tube. The frame 3010 and headgear tube(s) may together be referred to as a substructure 3750.

In the example shown the frame 3010 defines an opening 3020 in a superior face 3030 thereof. In examples, the opening 3020 extends to a posterior face 3040 of the frame 3010.

As is described above, the flexible cover 3310 may comprise a seal forming structure comprising a seal forming portion 3180 which forms a seal around at least an entrance to the patient's nares. The frame 3010 and flexible cover 3310 may be configured such that when the flexible cover 3310 is installed over the frame 3010, the frame 3010 holds the seal forming portion 3180 of the cover taut (e.g. with no or minimal sagging) across the opening 3020. The seal forming portion 3180 may comprise at least one hole for supplying air to the patient's nares. In examples, the seal forming portion 3180 comprises a plurality of holes, for example ten or more holes, as described further herein with reference to FIGS. 41A to 41D.

The portion of the frame 3010 surrounding the perimeter (e.g. the rim 3022) of the opening 3020 may be configured to be resiliently flexible. In examples, the rim 3022 comprises at least one. (e.g. a plurality of) slits 3024. The slits 3024 may extend substantially orthogonally to the perimeter of the opening 3020 (e.g. orthogonal to a tangent to the curve of the opening), although other angles may be used for some of the slits 3024. For example, the slits may form an angle of between 70 degrees and 90 degrees to the perimeter of the opening 3020. Other angles may also be useful. The slits 3024 may be configured to allow each portion 3026 of the rim 3022 to flex independently of the other portions 3026, for example when tension is created in the flexible cover 3310 due to engagement of the flexible cover 3310 with the patient's nose. In examples, the portions 3026 of the rim 3022 operate as independent cantilever spring elements. In other examples the material of the rim 3022 may be sufficiently flexible that such slits 3024 are not required.

The frame 3010 may also comprise posteriorly extending support portions 3028 configured to bring the seal forming portion 3180 of the flexible cover 3310 into firm engagement with the regions of the patient's face proximate the alar creases, in order to ensure adequate sealing in these areas. The frame 3010 may comprise one or more internal structures to increase its stiffness at or near the posteriorly extending support portions 3028, for example internal ribs 3042. In other examples the posteriorly extending support portions 3028 may be made from stiffer and/or thicker material than the material of the rim 3022. In some examples the use of stiffer or thicker material may mean that ribs 3042 are not necessary.

In examples, the portion of the flexible cover 3310 comprising the seal forming portion 3180 may be resiliently extensible. In examples, the seal forming portion 3180 may “balloon” under pressure (e.g. therapy pressure), thereby increasing contact with the patient's face. The resilience of the material may mean that it can more closely conform to creases and undulations in the patient's face. Portions of the seal forming portion 3180 may be coated with silicone while the flexible cover is inverted, e.g. while the diffuser material and/or the heat exchanger material are being connected to the flexible cover.

In examples, the substructure 3750 (e.g. the frame 3010 and/or tubes 3320) may comprise an indexing structure 3324 configured to engage the flexible cover 3310 and thereby ensure that the seal forming portion 3180 is correctly orientated relative to the frame 3010.

In examples the indexing structure 3324 comprise mushroom shaped protuberances provided to the tubes 3320. The protuberances may engage suitably sized and positioned slits or apertures 3326 in the flexible cover 3310. In examples, the slits or apertures 3326 are sized to closely conform to the size of the protuberances to thereby position the seal forming structure relative to the frame 3010 with a high degree of accuracy. In the example shown the head of the protuberance 3324 is substantially oval shaped.

Indexing structures 3324 having other shapes, e.g. substantially cylindrical or rectangular cross-section protruding portions, may also be used.

As is noted above, HME material 3510 may be provided to an internal surface of the flexible cover 3310, in the region of the seal forming portion 3180.

Diffuser material 3412 may also be provided to an internal surface of the flexible cover 3310 to overlie the vent 3400.

In the example shown in FIG. 42, both tubes 3320 may be formed together, as described in more detail above. A cushion module 3950 may be provided which is connectable to the tubes 3320. The cushion module 3950 may be provided with a rigid or semi-rigid connector portion 3962 which is engageable with an aperture 3328 in the tube(s). In examples, the tubes 3320, including the perimeter of the aperture 3328, are formed from silicone or a similarly stretchy material.

### 4.3.12.1.1 Textile Conduits and Seal

Referring next to FIGS. 25-28, in another example of the technology a textile structure 3430 defines at least one headgear tube 3320 and a seal forming structure 3100. The textile structure 3430 further defines, at least in part, a plenum chamber 3200.

Examples of the textile structure 3430 may appear similar to, and may be manufactured with similar methods to, the flexible cover 3310 described elsewhere herein. However, in the example shown in FIGS. 25-28, rather than the textile forming a cover for an underlying air-tight substructure, the textile structure 3430 may comprise an internal coating or lining 3432 (e.g. of silicone) configured to make the textile structure 3430 itself substantially air-tight, as shown in FIG. 26A. In examples the flexible cover may be coated by the method described below with reference to FIGS. 33-40.

The textile structure 3430 may comprise an opening 3440 in an anterior wall thereof. The opening 3440 may be sized to allow an undercushion structure 3900 to be inserted into the plenum chamber 3200 in order to support the lateral and anterior edges of the seal forming structure 3100. In examples, the textile structure 3430 may comprise a thickened rim 3450 of silicone around the opening 3440 to allow for stretch fit engagement with a vent module 3410.

As with the embodiment described above with reference to FIGS. 18-24, the undercushion 3900 may be configured to ensure that the portion 3180 of the textile forming the seal forming structure 3100 is held substantially taut when not in contact with the patient. The undercushion 3900 may comprise an opening 3020 in a superior face, as with the example described above.

The seal forming portion 3810 of the textile may also be configured to “balloon” under pressure, as described above.

The undercushion 3900 may also comprise posteriorly extending portions 3028 configured to bring the seal forming portion 3180 of the textile structure 3430 into firm engagement with the regions of the patient's face proximate the alar creases in order to ensure adequate sealing in these areas.

In examples the undercushion 3900 is made from, for example, an integral skin polyurethane foam or other suitable non-porous foam material.

In examples, the undercushion 3900 comprises a chassis portion 3910 and an HME portion 3500. The HME 3500 may be provided to a posterior side of the chassis portion 3910. A superior surface of the HME 3500 is configured to be below the rim of the opening 3020 in the chassis portion 3910, to allow the seal forming structure 3100 to deflect downward under pressure from the patient's nose.

In examples, the chassis portion 3910 of the undercushion 3900 comprises a plurality of channels 3920 therein. The channels 3920 may be configured to allow flow from the seal forming portion 3180 through the plenum chamber 3200 to a vent module 3410 provided in the opening. The channels 3920 may also be configured to allow air from the headgear tubes 3320 to enter the plenum chamber and travel to the patient and to the vent.

In examples having an HME 3500, the channels 3920 may be configured to allow flow from the headgear tubes 3320 to flow to the vent 3400 without passing through the HME 3500, as shown in FIG. 28. This may assist with avoiding or reducing drying of the HME 3500 during breath pause.

In examples, the HME 3500 may be constructed from the same foam as the undercushion 3900. The HME 3500 and undercushion 3900 may be integrally formed.

In examples the chassis portion 3910 of the undercushion 3900 may be made from a skinned foam so that substantially no flow passes through the material of the chassis portion 3910 (except through any channels provided).

In examples, the undercushion 3900 can be removed from the textile structure 3430, for example to allow washing of the interior of the structure and/or to allow washing, inspection or replacement of the undercushion/HME.

In examples, undercushions 3900 may be manufactured in a variety of different sizes and/or shapes to fit patients with different facial geometries. In examples, a single sized/shaped textile structure 3430 can be used with any one of a plurality of different sized and/or shaped undercushions, since the textile structure 3430 may stretch to conform to the different undercushion configurations.

In examples, the textile structure 3430 may be formed by a suitable technique such as circular knitting or 3D knitting. In one form, the internal surface of the textile structure 3430 may be coated with a suitable lining material 3432 (e.g. silicone, thermoplastic elastomer (TPE), nitrile rubber or other suitable polymer material) by, for example, a rotational moulding technique. Other suitable techniques may also be used to coat the textile structure, for example the method described below with reference to FIGS. 33-40.

The presence of the lining material 3432 may make the textile structure substantially air-tight, and may create a cleanable internal surface. The lining material may also make textile structure 3430 more resilient, thereby assisting it to remain open for air flow.

### 4.3.12.1.2 Textile Cushion Module

Referring next to FIGS. 29-32, in another example of the technology a patient interface comprises a frame 3010 and at least one (e.g. two) headgear tubes 3320 formed integrally with the frame 3010. For example, the frame 3010 and the at least one headgear tube are formed as a unitary structure that is created as one piece or a single unit having no seams between the frame 3010 and the at least one headgear tube.

A cushion module 3950 is provided which is configured for removable connection to the frame 3010. The cushion module 3950 comprises an undercushion 3900.

In one example the undercushion 3900 is constructed from silicone. In other examples the undercushion 3900 may be constructed from foam, or a combination of silicone and foam. In the example shown the undercushion 3900 is made from a foam such as integral skin polyurethane foam or other suitable non-porous foam material, with a semi-rigid rim made from a suitable thermoplastic, e.g. Hytrel™. The cushion module 3950 may also comprise an HME 3500, as described above.

The cushion module 3950 may further comprise a seal forming structure 3100 provided over the undercushion 3900. In examples the seal forming structure 3100 comprises a silicone membrane, although the seal forming structure 3100 may also comprise a textile. The undercushion 3900 may be configured to hold the seal forming structure 3100 taut when not in use, and to balloon under pressure, as with the examples described above with reference to FIGS. 18-24 and/or FIGS. 25-28. As with those examples, the undercushion 3900 may be configured to support the lateral and anterior edges of the seal forming structure 3100, but not an area between those edges.

The undercushion 3900 may also comprise posteriorly extending support portions 3028, similar to those examples referred to above.

The cushion module 3950 may further comprise a vent 3400 on an anterior side thereof.

In examples the posterior side of the frame 3010 defines a concave shape which is configured to be complementary to a convex anterior side of the cushion module 3950. In examples the frame 3010 may be generally U shaped. The frame 3010 may comprise diffuser material 3412 and/or at least one aperture to allow flow from the vent 3400 to escape to atmosphere. In examples, the cushion module 3950 may also comprise a diffuser material 3412 on an interior side of the vent.

The cushion module 3950 comprises at least one (e.g. two) inlet ports 3960. The inlet ports 3960 may be configured to receive a flow of breathable gas from the headgear tubes 3320. In examples, the frame 3010 may comprise a boss portion 3970 for each headgear tube, each boss 3970 provided to an inwardly facing surface of the frame 3010. Each boss portion 3970 may comprise an aperture 3980 therethrough to allow flow from the headgear tube 3320 to flow through the boss 3970. Each boss portion 3970 may be configured to engage a respective inlet port 3960 of the cushion module 3950. The boss portion 3970 and/or the inlet port 3960 may be provided with sealing means, for example lip seals 3990.

The boss portions 3970 may operate to secure the cushion module 3950 to the frame 3010, in use.

In another example the cushion module 3950, including the seal forming structure 3100, may be made from foam, e.g. integrally formed from foam.

Other forms of cushion module 3950 are shown in FIGS. 42, 42A and 43A-43E. In these examples, the cushion module 3950 comprises a foam chassis 3910 which is covered in a textile seal forming structure 3100. The foam chassis 3910 may be made from heat and moisture exchange material 3510, from a suitable alternative foam or from a mouldable material such as silicone. The cushion module may be provided with a rigid- or semi-rigid connector portion for engaging a tube, as described further herein.

In one example, a cushion module 3950 is made by the following steps.

As shown in FIG. 43A, a foam laminate 7200 is provided comprising a textile layer 7210 connected to a foam substrate 7220 by an airtight layer 7230 (e.g. made from thermoplastic polyurethane). The layers may be connected by, for example, hotmelt lamination. An aperture 7240 is provided through the laminate 7200. The aperture 7240 is sized to allow sufficient airflow for the patient to breathe during therapy.

As shown in FIG. 43B, a textile laminate 7250 is provided comprising a textile layer 7260 and an airtight layer 7270 (e.g. thermoplastic polyurethane). The layers may be connected by, for example, an adhesive. The textile laminate 7250 may be provided with at least one aperture 3150 to allow airflow to the patient's nares, where the aperture(s) 3150 are sized such that together they allow sufficient airflow for the patient to breathe during therapy. As is discussed further above, more than one aperture 3150 may be provided. For example, multiple apertures 3150 may be provided for each naris.

The textile laminate 7250 may be brought together with the foam laminate 7200 such that the textile layers 7210, 7260 of each laminate are in contact. The two laminates 7200, 7250 may then be cut to shape using RF die cutting. The RF die cutting process may not only cut the two laminates to a required shape, it may also weld the two airtight layers 7230, 7270 together at their outer perimeter. One form of the technology shown after the RF cutting step is shown in FIG. 43C.

Next, the outer textile layer (e.g. the textile layer 7260 of the textile laminate 7250) may be pulled through the aperture 7240 to form a cushion 7280 with a textile 7260 on its outer surface, as show in FIG. 43D.

Referring next to FIG. 43E, a rigid connector portion 3962 may be connected to the cushion 7280 (e.g. by overmoulding or gluing) to allow the cushion module 3950 to be connected to a frame or tube. The connector portion 3962 may comprise an engaging face 7290 with a curved profile. The cushion 7280 may adopt this curvature when connected to the engaging face 7290, and may thereby be held in a required “cradle” shape to engage the inferior surface of the patient's nares.

### 4.3.13 Method of Coating a Textile

Referring next to FIGS. 33-40, a method of coating an internal surface of a textile component is shown. While the method is described below with reference to the manufacture of a tube 3320, it should be appreciated that many components can be manufactured using the method, including, for example, a flexible cover as described above or a combination of one or more tubes and flexible cover.

In a first step, the textile outer layer 3432 of the tube is manufactured. The textile outer layer 3432 may be considered a “blank”. In examples, the outer layer 3432 may be manufactured in one piece to shape, for example by a 3D or circular knitting process. In examples the outer layer 3432 may be seamless. However, in other forms of the technology the outer layer 3432 may be made by connecting a plurality of textile components together, e.g by RF welding.

The outer layer 3432 may then be inserted into a female mould 6000, for example a two-piece female mould 6000 as shown in FIGS. 33, 34 and 36. The mould 6000 may be configured to hold the outer layer 3432 in a required shape, for example by means of a plurality of vacuum holes 6010 distributed over the inner surface 6020 of the mould. The vacuum holes 6010 may be connected to a vacuum pump, e.g. via a manifold and/or suitable conduits 6030. Those skilled in the art will appreciate that the term “vacuum” is used here to denote any suitable air pressure which is less than ambient air pressure, and is not limited to a pressure of absolute zero.

With the outer layer 3432 held in shape in the mould 6000 by the vacuum, a nozzle 6040 may spray a settable material 6050 such as silicone onto the interior of the flexible cover 3432 to create an air-tight seal. In examples, the nozzle 6040 may be provided at the end of a flexible tube 6070 to allow the nozzle 6040 to follow the shape of the component in the mould 6000 (e.g. a curved tube shape as shown in FIGS. 33 and 34. As shown in FIGS. 34 and 35A-35E, the nozzle 6040 may be inserted through the component before any material is passed through the nozzle 6040. The nozzle 6040 may then be drawn back out of the component with the nozzle 6040 spraying the silicone or other material 6050. The nozzle 6040 may spray in a direction generally away from the path of the nozzle 6040, in order to avoid the nozzle being dragged through the recently sprayed coating material 6050. In other examples (e.g. for manufacture of substantially straight tubes and/or masks and the like) the spray nozzle 6040 may be provided to the end of a substantially rigid tube.

In examples, the nozzle 6040 may be provided with spacing means (not shown) to hold the nozzle a required distance from the lower surface of the outer layer 3432 as it is drawn over the outer layer 3432 during the spraying process.

The inner coating 6060 may be formed by several passes of the nozzle 6040 (e.g. allowing each previous coating to at least partially cure before re-coating) in order to increase the wall thickness of the coating 6060. FIG. 36 shows a cross-section of the component in the mould 6000 after coating.

Because the outer surface of the outer layer 3432 is held firmly in place against the mould 6000 during the spraying operation, the outer surface may remain free of any overspray from the nozzle 6040.

As shown in FIG. 37, when the settable material 6050 is sufficiently set the pressure in the vacuum holes 6010 is allowed to return to ambient pressure and the component is removed from the mould 6000. However, the component may maintain the shape created by the mould 6000. The finished component may conform closely to the shape of the mould 6000, even if the outer layer 3432 is not manufactured with a high degree of dimensional accuracy, as may sometimes be the case with some textile manufacturing processes.

In examples, the mould 6000, with the component still inside, may be heated (e.g. in an oven) to assist with curing the internal coating 6060, prior to removing the component from the mould.

FIG. 38 shows a similar mould 6000 used to manufacture a corrugated tube 3320a. As shown in FIGS. 39 and 40, once the outer layer/blank 3432 has been coated with the inner layer 6060, it may continue to hold the form created by the mould 6000. For example, a corrugated tube 3320a may be formed using a non-corrugated textile blank 3432.

An inner silicone layer created by the method described above may provide support to the textile layer 3432, such that in some forms of the technology a patient interface may be formed from a textile with the silicone coating forming an integral frame or substructure.

In examples formed by this method, there is no need to invert a flexible cover during the manufacture of the patient interface, as is the case with the example shown in FIGS. 18-24. In particular, it is not necessary to invert the flexible cover after the silicone has been applied.

In examples, a flexible cover may include tube portions which are made air-tight by the application of silicone, as described above. In some such examples, no part of the frame or headgear tube has a Shore A Durometer hardness greater than 80, e.g. no greater than 60. However, in other examples the frame may be configured with connectors for connecting to one or more separate tubes, for example tubes made from silicone with no textile covering.

HME and/or diffuser material may be provided to the flexible cover, as described above.

### 4.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., filters 4110 such as an inlet air filter 4112 and outlet air filter 4114, an inlet muffler 4122, a pressure generator 4140 capable of supplying air at positive pressure (e.g., a blower 4142 comprising a motor 4144), a muffler 4120 such as an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices, a central controller, a therapy device controller, a pressure generator 4140, one or more protection circuits, memory, transducers 4270, data communication interface and one or more output devices. Electrical components 4200 may be mounted on a single Printed Circuit Board Assembly (PCBA) 4202. In an alternative form, the RPT device 4000 may include more than one PCBA 4202.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 4.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

In examples the humidifier reservoir further comprises a conductive portion 5120, a locking lever 5135 and a water level indicator 5150.

In one form of the present technology, an anti-spill back valve 4160 is located between the humidifier 5000 and the pneumatic block 4020.

In some forms of the technology an RPT for use with a patient interface of the present technology may not require a humidifier.

### 4.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6s, peak inspiratory flow rate Qpeak 0.4 L/s, exhalation time Te 2.4s, peak expiratory flow rate Qpeak-0.5 L/s. The total duration of the breath, Ttot, is about 4s. The person typically breathes at a rate of about 15 breaths per minute (BPM), with Ventilation Vent about 7.5 L/min. A typical duty cycle, the ratio of Ti to Ttot, is about 40%.

### 4.8 GLOSSARY

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.Patient: A person, whether or not they are suffering from a respiratory condition.

Pressure: Force per unit area. Pressure may be expressed in a range of units, including cmH2O, g-f/cm² and hectopascal. 1 cmH₂O is equal to 1 g-f/cm² and is approximately 0.98 hectopascal (1 hectopascal=100 Pa=100 N/m²=1 millibar˜ 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.8.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.8.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

4.8.2 Anatomy

### 4.8.2.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 4.8.2.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.8.3 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.8.4 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 4.8.4.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 4.8.4.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 4.8.4.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 4.8.4.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 4.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.10 REFERENCE SIGNS LIST

patient 1000
bed partner 1100
patient interface 3000
frame 3010
opening 3020
rim 3022
slits 3024
rim portion 3026
posteriorly extending support portions 3028
superior face 3030
posterior face 3040
internal ribs 3042
seal forming structure 3100
textile seal 3110
seal support portion 3120
foam seal 3130
tab 3132
silicone seal 3134
non patient contacting side 3136
adhesive 3140
adhesive tape 3142
hook and loop fastener portion 3144
hook material 3145
loop material 3146
snap fastener 3148
apertures 3150
strip 3152
perimeter of aperture 3160
aperture in seal support portion 3170
flexible lip 3172
seal forming portion 3180
plenum chamber 3200
chord 3210
superior point 3220
inferior point 3230
inlet port 3240
positioning and stabilising structure 3300
flexible cover 3310
end of cover 3312
tube 3320
corrugated tube 3320a
manifold portion 3322
manifold component 3323
connector portions 3323a
indexing structure 3324
slits 3326
aperture 3328
back strap 3330
tab 3340
vent 3400
vent module 3410
diffuser material 3412
aperture 3420
textile structure 3430
textile outer layer/blank 3432
opening 3440
opening rim 3450
HME 3500
arm portions 3502
HME base portion 3504
portions 3506
HME material 3510
connection port 3600
elbow 3610
forehead support 3700
substructure 3750
patient interface 3800
undercushion 3900
chassis portion 3910
channels 3920
cushion module 3950
inlet port 3960
connector portion 3962
boss 3970
aperture 3980
lip seal 3990
RPT device 4000
external housing 4010
upper portion 4012
lower portion 4014
panel 4015
chassis 4016
handle 4018
pneumatic block 4020
air filter 4110
outlet muffler 4124
pressure generator 4140
blower 4142
motor 4144
anti-spillback valve 4160
air circuit 4170
supplementary gas 4180
electrical components 4200
PCBA 4202
electrical power supply 4210
input device 4220
transducers 4270
humidifier 5000
humidifier inlet 5002
humidifier outlet 5004
humidifier base 5006
humidifier reservoir 5110
conductive portion 5120
reservoir dock 5130
locking lever 5135
water level indicator 5150
heating element 5240
female mould 6000
vacuum holes 6010
mould inner surface 6020
vacuum conduit 6030
nozzle 6040
settable material 6050
inner coating 6060
flexibe tube 6070
prong receiving apertures 7000
pillow prongs 7010
sealing face 7012
base component 7020
prongs module 7030
neck portion 7040
textile cover 7100
prong engaging apertures 7110
apertures 7120
foam laminate 7200
textile layer 7210
foam substrate 7220
airtight layer 7230
aperture 7240
textile laminate 7250
textile layer 7260
airtight layer 7270
cushion module 7280
engaging face 7290

1.-74. (canceled)

75. A patient interface comprising a frame which defines, at least in part, a plenum chamber, and a positioning and stabilising structure comprising at least one headgear tube, wherein the frame, at least one headgear tube and manifold portion are integrally formed.

76. The patient interface of claim 75, wherein the patient interface comprises a seal support portion which is formed integrally with the frame.

77. The patient interface of claim 76, wherein the patient interface is provided with a seal portion, wherein the seal portion is connected to the seal support portion.

78. The patient interface of claim 77, wherein the seal portion is releasably connected to the seal support portion.

79. The patient interface of claim 77, wherein the seal portion comprises foam or silicone.

80. The patient interface of claim 77, wherein the seal portion comprises a textile.

81. The patient interface of claim 77, wherein the seal portion is connected to the seal support portion by adhesive, at least one hook and loop fastener, or at least one snap fastener.

82. The patient interface of claim 75, wherein the patient interface comprises a seal forming structure which is formed integrally with the frame.

83. The patient interface of claim 82, wherein the seal forming structure comprises a textile layer.

84. The patient interface of claim 83, wherein the textile layer is provided with a silicone backing layer.

85. The patient interface of claim 75, wherein the patient interface comprises a flexible cover which covers at least a portion of each tube.

86. The patient interface of claim 85, wherein the flexible cover comprises a textile.

87. The patient interface of claim 85, wherein the flexible cover comprises at least one opening configured to allow inspection of the tube through the opening.

88. The patient interface of claim 85, wherein the flexible cover comprises at least one strap connecting structure for connecting a back strap to the flexible cover.

89. The patient interface of claim 85, wherein the flexible cover comprises a back strap.

90. The patient interface of claim 85, wherein a back strap is integrally formed with the flexible cover.

91. The patient interface of claim 75, wherein the positioning and stabilising structure comprises a pair of headgear tubes, and wherein a back strap is integrally formed with the headgear tubes.

92. The patient interface of claim 75, wherein the positioning and stabilising structure comprises a pair of headgear tubes, and wherein the manifold portion comprises a pair of upper tube portions, each upper tube portion comprising a concertina formation.

93. The patient interface of claim 75, wherein the patient interface comprises a vent module, wherein the vent module is configured to engage the frame in a stretch fit.

94. The patient interface of claim 75, wherein the patient interface comprises a vent module and a heat and moisture exchanger connected to the vent module.
